(19) United States
(12) Patent Application Publication
(10) Pub. No.: US 2026/0002935 A1
(43) Pub. Date: Jan. 1, 2026

(54) CHEMICAL SENSORS EMPLOYING pH-SENSITIVE APTAMERS

(71) Applicant: Ohio State Innovation Foundation, Columbus, OH (US)

(72) Inventors: Jinghua LI, Columbus, OH (US); Shulin CHEN, Columbus, OH (US); Tzu-Li LIU, Columbus, OH (US)

(21) Appl. No.: 18/992,247

(22) PCT Filed: Jul. 10, 2023
(86) PCT No.: PCT/US2023/027293

§ 371 Date: Jan. 8, 2025

Related U.S. Application Data
(60) Provisional application No. 63/359,734, filed on Jul. 8, 2022.

(51) Int. Cl. G01N 33/543; C12N 15/115; G01N 33/94

(52) U.S. Cl. CPC G01N 33/5438; C12N 15/115; G01N 33/9413; G01N 33/942; G01N 33/946; C12N 2310/16

(57) ABSTRACT

Described herein are sensors for detecting an analyte of interest in a sample. These sensors can comprise a potentiometric sensor comprising a surface functionalized with a pH-sensitive aptamer switch that specifically binds the analyte of interest, wherein the pH-sensitive aptamer switch is operatively coupled to the potentiometric sensor such that binding of the analyte of interest by the pH-sensitive aptamer switch induces a measurable change in the potentiometric sensor; and an auxiliary electrode in proximity to the surface, wherein the electrode is configured to alter a pH of a microenvironment in contact with the surface, thereby reversibly shuttling the pH-sensitive aptamer switch between a first state wherein it specifically binds the analyte of interest and a second state wherein it does not specifically bind the analyte of interest.

### CROSS REFERENCE TO RELATED APPLICATIONS

This application claims the benefit of U.S. Provisional Application No. 63/359,734, filed Jul. 8, 2022, which is incorporated herein by reference in its entirety.

### BACKGROUND

One common goal shared by research in biosensing, analytical chemistry, and bio-integrated electronics is to develop designs and integration schemes for sensors that support quantitative and real-time monitoring of biomarker concentrations in multiple bodily fluids such as saliva, blood, interstitial fluids, urine, and others. Conventional biosensing techniques, such as enzyme-linked immunosorbent assay (ELISA), capillary electrophoresis (CE), gas chromatography (GC), and high-performance liquid chromatography (HPLC), require the use of centralized equipment and trained personnel for operation, and thus are not compatible with continuous monitoring in home, community, and workplace settings. To this end, label-free biosensors with functionalized biochemical interfaces provide a realistic route for the rapid detection of biomarkers in complex environments. Nevertheless, the bottleneck for continuous monitoring using biosensor chips is in the reusability of the immobilized bio-recognition elements.

Pioneering studies report the regeneration of biochemical sensors through regrafting biorecognition elements after usage. These methods, however, rely on additional chemical reagents for surface cleaning followed by re-functionalization of the devices and therefore, are not compatible with building in vivo biosensors for continuous monitoring. Removal of adsorbed biomarkers through electrochemical reactions/electrical repulsion serves a promising strategy for in situ regeneration but may require delicate design of the sensing interfaces consisting multiple functional layers such as molecularly imprinted polymers and redox-active nanoreporters. In-vitro selected aptamers enabled by the systematic evolution of ligands by exponential enrichment (SELEX), on the other hand, provide attractive opportunities for developing reusable sensors due to the reconfigurable nature of DNA sequences. However, while their noncovalent interactions with the corresponding targets (e.g., hydrogen bonds, electrostatic bonds, Van der Waals forces) are reversible in nature, conventional methods for dissociating targets from aptamers exploit extreme conditions, such as with heating, ultraviolent light exposure, and concentrated chemicals.

Consequently, most biosensors still have a limited capability for repetitive use due to the surface saturation issue. The knowledge gap highlights the crucial need for establishing general sensing schemes that allow for the regeneration of biosensor surfaces and continuous monitoring of biomolecules.

### SUMMARY

Electrochemical sensors for the detection and quantification of analytes are provided. The sensors can be used to accurately and rapidly detect and/or quantify analytes of interest. The sensors described herein employ aptamers (e.g., pH-sensitive aptamer switches) that exhibit a high binding affinity towards an analyte of interest. The aptamers can thus serve as the sensing interface of the electrochemical sensors. When pH-sensitive aptamer switches are employed, the sensor can possess an allosteric nucleic acid-functionalized surface that exhibits pH-tunable performance via a potentiometric sensing strategy. For example, the sensor can comprise a FET-based potentiometric sensor that includes an allosteric nucleic acid-functionalized surface that exhibits pH-tunable performance. Following analyte binding, sensing capability can be restored by altering the pH of the medium in contact with the sensor (first decreasing binding affinity for the analyte of interest, causing it to be released, followed by restoring pH to increase affinity, thereby restoring affinity for the analyte of interest). pH can be controlled using an electrode (e.g., a Pd electrode) positioned in proximity to the nucleic acid-functionalized surface. This electrode can be used to reversibly and focally control the pH of the microenvironment in contact with the nucleic acid-functionalized surface, allowing for sensor regeneration to be performed with minimal perturbation of the surrounding medium.

These sensors can be inductively coupled to a signal transducer, a transmitter, or a combination thereof to produce a wireless sensor. As such, these sensors can be flexible, biocompatible, lightweight, and/or implantable, allowing them to be deployed in a variety of applications (e.g., regeneratable wearable electronics, bioimplants, point of care diagnostics, etc.). In some embodiments, these sensors can provide for wireless, regeneratable chemical sensing of analytes within a sample or medium (e.g., a biological sample such as a biofluid). In some embodiments, these sensors can be used to detect small molecules of biochemical relevance, such as cocaine, dopamine, serotonin, etc.

For example, provided herein are sensors for detecting an analyte of interest in a sample. These sensors can comprise a potentiometric sensor comprising a surface functionalized with a pH-sensitive aptamer switch that specifically binds the analyte of interest, wherein the pH-sensitive aptamer switch is operatively coupled to the potentiometric sensor such that binding of the analyte of interest by the pH-sensitive aptamer switch induces a measurable change in the potentiometric sensor; and an auxiliary electrode in proximity to the surface, wherein the electrode is configured to alter a pH of a microenvironment in contact with the surface, thereby reversibly shuttling the pH-sensitive aptamer switch between a first state wherein it specifically binds the analyte of interest and a second state wherein it does not specifically bind the analyte of interest.

In some embodiments, the pH-sensitive aptamer switch can be covalently conjugated to the surface of the potentiometric sensor.

In some embodiments, the surface can comprise an electrode surface. For example, in some embodiments, the potentiometric sensor can comprise a working electrode (or sensing electrode) and a reference electrode, and the surface of the working electrode can be functionalized with the pH-sensitive aptamer switch that specifically binds the analyte of interest. In certain embodiments, the potentiometric sensor can comprise a working electrode (or sensing electrode) and a reference electrode, and the pH-sensitive aptamer switch can be covalently conjugated to the surface of the working electrode.

In other embodiments, the potentiometric sensor can comprise a field-effect transistor (FET). For example, the sensor can comprise a substrate and a channel that is disposed on the substrate. The sensor can further include a source electrode and a drain electrode electrically connected to the channel. The source electrode and the drain electrode can be formed to be separate such that the channel forms a path for current flow between the source electrode and the drain electrode. In these embodiments, the surface of the channel can be functionalized with the pH-sensitive aptamer switch that specifically binds the analyte of interest. In certain embodiments, the pH-sensitive aptamer switch can be covalently conjugated to the surface of the channel.

In some embodiments, the auxiliary electrode can comprise a Pd electrode. In some examples, the potentiometric sensor can comprise a working electrode (or sensing electrode) and a reference electrode, and the auxiliary electrode can circumferentially disposed around the working electrode and the reference electrode. In other examples, the potentiometric sensor can comprise a field-effect transistor, and the auxiliary electrode can circumferentially disposed around the field-effect transistor.

In some embodiments, the potentiometric sensor can be inductively coupled to a signal transducer, a transmitter, or a combination thereof.

The analyte of interest can comprise any target molecule of interest that can be recognized by the pH-sensitive aptamer switch. In some embodiments, the pH-sensitive aptamer can exhibit a binding affinity for the analyte of interest in the first state that is at least 10, 50, 100, 250, 500, or 1000 times greater than a binding affinity for the analyte of interest in the second state.

In some examples, the analyte of interest can comprise a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or biomolecule.

In certain embodiments, the analyte of interest comprises a biomolecule.

In certain embodiments, the analyte of interest comprises a small molecule (e.g., an organic molecule having a molecular weight of less than 1,000 Da, less than 800 Da, or less than 500 Da).

In certain embodiments, the analyte of interest comprises a drug, such as cocaine.

In certain embodiments, the analyte of interest comprises a neurotransmitter, such as serotonin or dopamine.

In some embodiments, the sensors described herein can be biocompatible, implantable, flexible, or any combination thereof. These sensors can be utilized in a wide variety of sensing applications, including biosensing applications. In some embodiments, the sensors described herein can be integrated into a regeneratable wearable electronic device, a bioimplant, a point of care diagnostic, or any combination thereof.

Also provided are methods of detecting an analyte of interest in a medium that comprise contacting the medium with a sensor described herein. In some examples, the medium can comprise a biological sample, such as bodily fluid or tissue (e.g., in vivo or ex vivo).

### BRIEF DESCRIPTION OF THE FIGURES

FIG. 1. Regeneration mechanism of the pH sensitive anti-cocaine aptamer based on allosteric regulation. (A) DNA Sequence of the re-engineered anti-cocaine aptamer used in this study. The aptamer has a pH sensitive motif at the 5′ end enabling the reversible duplex-to-triplex switch through the formation of intramolecular Hoogsteen interactions in the acidic environment; (B) Schematic illustration of the conformational change of the pH sensitive aptamer labeled with a fluorophore and a quencher. The fluorescence intensity resulting from the interaction between the fluorophore and quencher serves as a measure for characterizing the conformation; (C) Fluorescence intensity of the labeled anti-cocaine aptamer responding to test solutions with varying pH values (4.0 to 7.4) and cocaine concentrations (10⁻¹¹ to 10⁻³ M); (D) Real-time fluorescence intensity of the aptamer to dynamic changes in pH values (cocaine concentration: 10⁻⁵ M) showing the fast response time.

FIG. 2. Design, preparation, and characterization of potentiometric anti-cocaine sensing platforms based on the pH sensitive aptamer. (A) Fabrication procedures for the aptamer-functionalized sensor for cocaine detection; (B) Open circuit potential of the resulting potentiometric sensors in 1×PBS solutions with different concentrations of cocaine ranging from 10⁻¹¹ M to 10⁻⁵ M (after signal stabilization); (C) EIS characterization (Nyquist plot) of a sensing electrode in 1×PBS solutions with different concentrations of cocaine; (D) Extracted charge transfer resistance based on results in FIG. 2C; (E) Calibrated response plots of sensing electrodes with different ratios of aptamer to MCH used for surface functionalization; (F) Extracted sensitivities of electrodes with different aptamer-to-MCH ratios based on results in FIG. 2E; (G) Comparison of responses of the anti-cocaine sensors to cocaine and potential interferents including glucose, atropine, serotonin, tropinone, BSA and urea.

FIG. 3. Characterization of the regeneration behavior of the pH sensitive aptamer sequence through allosteric regulation. (A) Working principle of the pH-induced sensing surface regeneration. At neutral pH, the anti-cocaine aptamer with pH sensitive motif (Aptamer 1) has an affinity to its target and can fold into a three-way junction upon binding. As pH decreases, the triplex formation at 5′ end disrupts the stem structure, inhibits binding, and facilitates the release of cocaine molecules. In contrast, the DNA sequence without the pH sensitive motif (Aptamer 2) lacks the capability of regeneration; (B) Calibrated response of the anti-cocaine sensors with Aptamer 1 (in mV) as a function of cocaine concentration in 1×PBS with different pH values. The curve at pH=7.4 uses the same data in FIG. 2E (aptamer to MCH ratio=1:100) for comparison; (C) Calibrated response plots of saturated anti-cocaine sensors regenerated in solution with pH values of 4.0, 5.0 and 6.0 for 60 min, respectively; (D) Calibrated response plots of saturated anti-cocaine sensors regenerated in 1×PBS solution (pH=5.0) for 30, 45, and 60 min, respectively. The curve for 60 min uses the same data in FIG. 3C (pH=5.0, 60 min) for comparison; (E, F, G) Sequential display of sensing performance of three anti-cocaine sensors before and after repetitive regeneration cycles (pH=5.0, 60 min); (H) Summary of average sensitivity extracted from FIG. 3E-G as a function of the number of regeneration cycle (0-7); (I) Sensing performance of anti-cocaine sensors functionalized with Aptamer 2 during repetitive regeneration cycles; (J) EIS characterization (Nyquist plot) of a sensing electrode with Aptamer 2 in 1×PBS solutions with different concentrations of cocaine.

FIG. 4. Design, fabrication, and characterization of Pd electrodes for local pH regulation. (A-C) Schematic illustration of preparing Pd electrodes as the pH regulator that can modify the concentration of protons in surrounding environment via electrochemical actuation; (D-E) SEM images of Au surface before (D) and (E) after deposition of PdNps (˜300 nm); (F) SEM image of PdHₓ after the loading of hydrogens; (G) EDS analysis of Au surfaces before/after electrodeposition confirming the formation of PdNps; (H) I-t curves recorded during the loading and release of protons into and from the Pd electrode; (I) Photographs showing changes in pH values of solutions collected near the working electrode before/after the loading/release processes; (J) Sequential photographs showing the dynamic pH change and spatial distribution of protons in solution during the loading and cyclic release processes. The arrows indicate the regions that are going through the color change; (K) Schematic illustration of the experimental setup for regeneration of an anti-cocaine using a Pd/PdH electrode under cyclic actuation shown in (J); (L) Calibrated response plots of the original and regenerated anti-cocaine sensors placed near the Pd electrode during the electrochemical actuation process.

FIG. 5. Design of bio-integrated electronic systems with wireless sensing/electrochemical actuation capabilities based on the regeneration scheme described in this work. (A) Schematic illustration of a “flower-shaped” sensor prototype integrated with an inductive coupling unit for wireless data transmission and RF energy harvesting, and envisioned applications of the bioelectronics (i.e., as wearable, implantable, and point-of-care devices); (B) Photographs of a wireless device in flat and bent configurations; (C) Equivalent circuits and flowcharts for the signal and power conversion-transmission processes during wireless operation of the device through inductive coupling.

FIG. 6. Characterization of the “flower-shaped” wireless electronic device. (A) Experimental setup for the cyclic stretch/release test using a tensile tester, and photographs of a device with a tensile strain ranging from 0 to 20% applied to the stretchable “stem”; (B) Change in fₛ of the sensor coil as a function of the applied tensile strain and stretch/release cycles; (C) Resonance curves of the coils for the sensor and pH regulator showing distinct values of fₛ obtained via proper design of the coil structures. The value of fₛ of the sensor coil becomes larger with an increasing input DC voltage serving as the reverse bias for diodes in the circuit; (D) Normalized values of measured S11 of an anti-cocaine sensor functionalized with the pH sensitive aptamer; (E) Extracted value of fₛ in (D) as a function cocaine concentration; (F) Experimental setup of the wireless electrochemical actuation system; (G) An RF energy radiated from the transmitter by applying an alternating current (sine wave), and the resultant output DC voltage signals wirelessly collected by the harvester (after rectification and voltage regulation); (H) Schematic illustration of the relative position between the harvester and transmitter coils; (I) Mapping of measured output voltages as a function of lateral displacement of the transmission coil from the origin (z=5 mm); (J) Recorded output voltage as a function of displacement of the transmission coil along the z-axis with and without the Zener voltage regulator and amplifier (x=y=0 mm).

FIG. 7. Calibrated response of the anti-cocaine sensors with aptamer 1 (in mV) as a function of cocaine concentration in 0.1×PBS with different pH values.

FIG. 8. Extended data for the selectivity study of the cocaine sensors. (A) Chemical structures of all the target and non-target analytes (except for BSA) used in this study; (B) Calibrated responses of the anti-cocaine sensors to non-target analytes including glucose, atropine, serotonin, tropinone, BSA and urea (the response to cocaine also included for comparison). The curve of cocaine uses the same data as in FIG. 2E.

FIG. 9. Regeneration behavior of anti-cocaine sensors functionalized with aptamer 1 in 1×PBS solution (pH=5.0) with agitation. The study uses the same group of sensors and conducts sequential regeneration for 5, 15 and 30 min. The data in this figure shows the sensing performance after each regeneration cycle.

FIG. 10. Regeneration behavior (pH=5.0, 60 min) of anti-cocaine sensors functionalized with aptamer 1 in 1×PBS solution with the cocaine concentration ranging from 10⁻¹¹ to 10⁻³ M. (A, B, C) Results for three independent samples; (D) Averaged values based on data in Figure A-C.

FIG. 11. Extracted charge transfer resistance based on results in FIG. 3(J).

FIG. 12. Regeneration performance of sensors functionalized with pH sensitive anti-streptavidin and anti-thrombin aptamers. (A) Working principle of the pH-induced sensing surface regeneration of an anti-streptavidin aptamer. At neutral pH, the anti-streptavidin aptamer with a folded conformation has an affinity to its target. As pH decreases to 5.0, a G-A mismatch plays a critical role in preventing the sequence from folding into the conformation for the interaction with streptavidin and thereby, enables the release of streptavidin; ¹(B) Calibrated response plots of the original and regenerated (pH=5.0, 60 min) anti-streptavidin sensors; (C) Position of mismatched bases, and structures of G-A and C-T mismatches due to pH change. Red dot: the pH-sensitive G-A mismatch predicted to stabilize the structure. Black dot: The C-T mismatch (not stabilized at acidic pH). Dashes: Watson-Crick base-pairs; (D) Working principle of the pH-induced sensing surface regeneration of an anti-thrombin aptamer. At neutral pH, the anti-thrombin aptamer with G-quadruplex has an affinity to its target. As pH decreases, a G-A mismatch interrupts the G-quadruplex and causes the release of thrombin; ²(E) Calibrated response plots of the original and regenerated (pH=5.0, 60 min) anti-thrombin sensors; (F) Transition in conformation between G-quadruplex and G-A mismatch in the anti-thrombin aptamer due to pH change.

FIG. 13. Cyclic voltammetry curves during the electrodeposition of PdNps (cycle 1-10).

FIG. 14. Quantification of hydrogen capacity in a Pd electrode. (A) I-t curve recorded during the processes of loading protons into a Pd electrode for 20 min; (B) Integration of current over time based on data in (A).

FIG. 15. Photograph showing changes in pH values of solutions collected near the working electrode before/after the loading/release processes.

FIG. 16. Raw data of measured S11 of an anti-cocaine sensor during mechanical testing: (A) with a tensile strain ranging from 0 to 20%, and (B) before/after 0-1000 stretch/release cycles.

FIG. 17. Raw data of measured S11 of the anti-cocaine sensor in FIG. 6(D) (before normalization).

FIG. 18. EIS characterization (Nyquist plot) of the Pd electrode 1×PBS solution.

FIG. 19. V-t plots of DC output collected from the receiver wirelessly upon systematically varying the relative position between the transmission and harvester coils within the (x, y) range from (−5,−5) to (5,5).

FIG. 20. Regeneration performance of a packaged “flower-shaped” wireless device. (A) Equivalent circuit of the wireless sensing part showing how to connect the coupling unit and sensing interface based on the polarity between SE and RE. One thing to note is that the baseline of OCP value (i.e., the OCP reading at the lowest concentration cocaine concentration for each sensitivity plot) may shift after the regeneration process due to changes in the surface states and local environment, which is a common issue for potentiometric sensors. Specifically for the example illustrated in this figure, the baseline value changes from negative to positive after regeneration. This requires switching the connection of SE and RE to the coupling unit for measurement, as this sensing circuit is designed to work with the input voltage serving as the reverse bias for the varactor diodes such that it will modify the thickness of the depletion region in the p-n junctions; (B) (C) Normalized values of measured S11 of an anti-cocaine sensor functionalized with the pH sensitive aptamers before and after regeneration. The connection of SE and RE to the coupling unit is switched for the characterization before/after regeneration to address the change in baseline OCP; (D) (E) Extracted values of fₛ based on resonance curves in (B) and (C) as a function cocaine concentration; (F) Calibrated response of the sensing interface in the packaged system calculated based on data in (D) and (E) (voltage sensitivity=0.042 MHz/mV).

FIG. 21. Autocad design of the inductive coupling units of an example “flower-shaped” sensor described in the Examples (zoom-in view: circuit layouts for the sensor and harvester coils).

FIG. 22. Perspective view of an example sensor described herein.

FIG. 23. Cross-sectional view of an example field-effect transistor that can serve potentiometric sensor component of the sensors described herein.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “aptamer” refers to any polynucleotide, generally an RNA or a DNA that has a useful biological activity in terms of biochemical activity, molecular recognition or binding attributes to a target. Usually, an aptamer has a molecular activity such as binding to a target at a specific binding site on the target. It is generally accepted that an aptamer which is specific in its binding to the target, may be synthesized and/or identified by SELEX. Aptamers can include two binding sites, a target binding site and a reporter binding site. In some embodiments, the aptamer can comprise a “pH-sensitive aptamer” (also referred to as a “pH-responsive aptamer,” a “pH-sensitive aptamer switch,” or a “pH-responsive aptamer switch”). Such aptamers switch binding affinity in a pH-responsive manner (e.g., such that the aptamers undergo a strong change in affinity—in acidic, neutral, or alkaline conditions). Such pH-sensitive aptamers can be generated using strategies known in the art, such as by inserting a known pH-sensitive DNA motif into the aptamer structure or by inserting two orthogonal motifs that can be manipulated in parallel to tune sensitivity to different pH conditions without altering the core sequence of the aptamer itself.

The term “systematic evolution of ligands by exponential enrichment” or “SELEX” generally means any method of selecting for an aptamer which binds to a target. SELEX involves screening a pool of random targets for a particular aptamer that binds to a target or has a particular activity that is selectable. Generally, the particular aptamer represents a very small fraction of the target pool, therefore, a round of aptamer amplification, usually via polymerase chain reaction, is employed to increase the representation of potentially useful aptamers. Successive rounds of selection and amplification are employed to exponentially increase the abundance of the particular and useful aptamer. SELEX is described in several publications including, but not limited to, Famulok, M.; Szostak, J. W., In Vitro Selection of Specific Ligand Binding Nucleic Acids, Angew. Chem. 1992, 104, 1001. (Angew. Chem. Int. Ed. Engl. 1992, 31, 979-988); Famulok, M.; Szostak, J. W., Selection of Functional RNA and DNA Molecules from Randomized Sequences, Nucleic Acids and Molecular Biology, Vol 7, F. Eckstein, D. M. J. Lilley, Eds., Springer Verlag, Berlin, 1993, pp. 271; Klug, S.; Famulok, M., All you wanted to know about SELEX; Mol. Biol. Reports 1994, 20, 97-107; and Burgstaller, P.; Famulok, M. Synthetic ribozymes and the first deoxyribozyme; Angew. Chem. 1995, 107, 1303-1306 (Angew. Chem. Int. Ed. Engl. 1995, 34, 1189-1192).

As used herein, the terms “antigen,” “target” and “analyte” are used interchangeably and refer generally to a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or any other agent which is capable of binding to an aptamer and thus capable of being detected using the sensors described herein. A target is characterized by its ability to be “bound” by the aptamer.

The term “antigen binding site,” “target binding site,” “analyte binding site,” or “epitope” refers to the portion of the target to which the aptamer binds.

The terms “bind,” “binds,” and “specifically binds” refers to the ability of an aptamer to bind to a target with greater affinity than it binds to a non-target. In certain embodiments, specific binding refers to binding for aptamer with an affinity that is at least 10, 50, 100, 250, 500, or 1000 times greater than the affinity for a non-target.

The term “binding affinity” refers to the strength of interaction between an aptamer and its target as a function of its association and dissociation constants. Higher affinities typically mean that the aptamer has a fast on rate (association) and a slow off rate (dissociation). Binding affinities can change under various physiological conditions due to changes that occur to the target or aptamer under those conditions. Binding affinities of the aptamer can also change when a reporter is attached. Binding affinities can also change when slight changes occur to the target, such as changes in the amino acid or nucleotide sequence or glycosylation of the target. Generally, the aptamers have high binding affinities for their respective targets (in the case of pH-sensitive aptamers, the aptamers generally exhibit a high binding affinity for their respective targets at at least one pH).

As used herein, the term “flexible” itself or when used to modify or describe the sensor and/or components thereof means capable of elastically bending or twisting under loads generated by body movements of the wearer of the sensor when generally in conformal contact with the wearer without disrupting sensor performance.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted subcutaneously (i.e., in the layer of fat between the skin and the muscle) or transcutaneously (i.e., penetrating, entering, or passing through intact skin), which may result in a sensor that has an in vivo portion and an ex vivo portion.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

### Sensors

Sensors for the detection and quantification of analytes are provided. The sensors can be used to accurately and rapidly detect and/or quantify an analyte of interest of interest in a sample. In certain embodiments, the sensors can accurately and rapidly detect and/or quantify an analyte of interest of interest under physiological conditions.

Provided herein are sensors for detecting an analyte of interest in a sample. These sensors can comprise a potentiometric sensor comprising a surface functionalized with a pH-sensitive aptamer switch that specifically binds the analyte of interest, wherein the pH-sensitive aptamer switch is operatively coupled to the potentiometric sensor such that binding of the analyte of interest by the pH-sensitive aptamer switch induces a measurable change in the potentiometric sensor; and an auxiliary electrode in proximity to the surface, wherein the electrode is configured to alter a pH of a microenvironment in contact with the surface, thereby reversibly shuttling the pH-sensitive aptamer switch between a first state wherein it specifically binds the analyte of interest and a second state wherein it does not specifically bind the analyte of interest.

Referring now to FIG. 22, in some embodiments, the surface can comprise an electrode surface. For example, in some embodiments, these sensors (100) can comprise a potentiometric sensor (101) that comprises a working electrode (or sensing electrode, 102) and a reference electrode (104). The surface of the working electrode (102) can be functionalized with the pH-sensitive aptamer switch that specifically binds the analyte of interest. In certain embodiments, the pH-sensitive aptamer switch can be covalently conjugated to the surface of the working electrode. The sensor can further comprise the auxiliary electrode (106) in proximity to the surface of the working electrode. In certain embodiments, the auxiliary electrode can circumferentially disposed around the working electrode and the reference electrode. In these embodiments, the electrodes can be fabricated from any suitable electrical conductors. Examples of suitable electrical conductors include, but are not limited to, gold, platinum, titanium, titanium carbide, tungsten, aluminum, molybdenum, chromium, tungsten silicide, tungsten nitride, and alloys and combinations thereof. The electrodes, alone and in combination, can be fabricated in any suitable orientation and geometry so as to facilitate sensor operation.

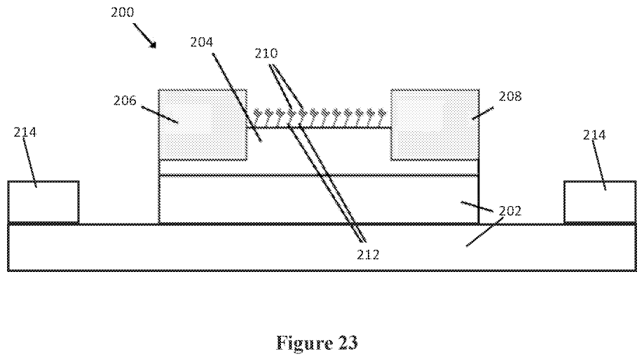

Referring now to FIG. 23, in other embodiments, the potentiometric sensor can comprise a field-effect transistor (FET). For example, the sensor (200) can comprise a substrate (202) and a channel (204) that is disposed on the substrate. The sensor can further include a source electrode (206) and a drain electrode (208) electrically connected to the channel. The source electrode and the drain electrode can be formed to be separate such that the channel (204) forms a path for current flow between the source electrode and the drain electrode. In these embodiments, the surface of the channel can be functionalized with the pH-sensitive aptamer switch (210) that specifically binds the analyte of interest. In certain embodiments, the pH-sensitive aptamer switch can be covalently conjugated to the surface of the channel (e.g., using a linking group 212). The sensor can further comprise the auxiliary electrode (214) in proximity to the surface of the channel. In certain embodiments, the auxiliary electrode can circumferentially disposed around the FET (e.g., around the surface of the channel).

In these embodiments, the electrodes can be fabricated from any suitable electrical conductors. Examples of suitable electrical conductors include, but are not limited to, gold, platinum, titanium, titanium carbide, tungsten, aluminum, molybdenum, chromium, tungsten silicide, tungsten nitride, and alloys and combinations thereof. The electrodes, alone and in combination, can be fabricated in any suitable orientation and geometry so as to facilitate sensor operation. Likewise, the channel can be fabricated from conventional materials used in FET channels, including semiconductors.

The field-effect transistor (FET) can optionally contain one or more additional components known in the art. For example, field-effect transistor (FET) can further comprise an insulator disposed on the source electrode, the drain electrode, or combinations thereof. The insulator can be configured to permit a conductive fluid to be applied to the surface of the channel without the conductive fluid completing a short circuit between the source electrode and the drain electrode. Insulators can also be disposed on a portion of the channel surface, for example, to create a well into which fluid samples can be applied.

The field-effect transistor (FET) can optionally include a gate electrode configured to apply a gate bias to the channel. A gate bias can be applied to the channel to allow the sensor to operate in the subthreshold regime. This can allow the sensor to be more sensitive to interaction of the aptamer with the analyte of interest. In some embodiments, the sensor is back-gated (i.e., it includes a gate electrode beneath the channel, such as within the substrate, which is configured to apply a gate bias to the channel). The sensor can include a side gate positioned adjacent to the channel, and configured to apply a gate bias to the channel. In some embodiments, a floating electrode in contact with the fluid in which the sensor is immersed is used to apply the gate bias.

In the sensors described herein, the pH-sensitive aptamer switch can be immobilized on the surface via a linking group, or by direct adsorption to the channel surface. In some embodiments, the pH-sensitive aptamer switch can be immobilized on the surface of the channel via a linking group. The linking group can be selected such that the distance between the pH-sensitive aptamer switch and the surface such that association of the analyte of interest with the pH-sensitive aptamer switch induces a change in the electronic properties of the potentiometric sensor. In some cases, the linking group is selected such that the distance between the pH-sensitive aptamer switch and the surface is less than about 10 nm (e.g., less than about 9 nm, less than about 8 nm, less than about 7 nm, less than about 6 nm, less than about 5 nm, less than about 4 nm, less than about 3 nm, less than about 2 nm, or less than about 1 nm).

In some embodiments, the linking group comprises a polyvalent linking group. Polyvalent linking groups are derived from polyvalent linkers (i.e., linkers which associate with the surface via two or more chemical moieties and have the capacity to be covalently or non-covalently linked to the pH-sensitive aptamer switch). For example, the polyvalent linking group can be derived from a small molecule linker that forms two or more covalent bonds with the surface and a covalent bond with the pH-sensitive aptamer switch.

In some embodiments where the linking group comprises a polyvalent linking group, the pH-sensitive aptamer switch is bound to an interfacial polymeric film, such as a silane polymer film derived from trialkoxysilane monomers. Examples of suitable polyvalent linking groups include thin films derived from polyvalent linkers including 3-aminopropyl)triethoxysilane (APTES), (3-glycidyloxypropyl) trimethoxysilane, (3-mercaptopropyl) trimethoxysilane, vinyltrimethoxysilane, allyltrimethoxysilane, (3-bromopropyl) trimethoxysilane, triethoxyvinylsilane, triethoxysilane aldehyde (TEA), and combinations thereof.

In certain embodiments, the linking group comprises a monovalent linking group. Monovalent linking groups are derived from monovalent linkers (i.e., linkers which associate with the surface via a single chemical moiety and have the capacity to be covalently or non-covalently linked to a pH-sensitive aptamer switch). For example, monovalent linking groups can possess a first moiety which is associated with or bound to the surface, and a second moiety which is associated with or bound to the pH-sensitive aptamer switch. In this way, the monovalent linker forms a molecular monolayer which tethers the pH-sensitive aptamer switch to the surface.

The monvalent linking group can be derived from a heterobifunctional small molecule which contains a first reactive moiety and a second reactive moiety. The first reactive moiety can be reactive with the surface (e.g., a thiol) and the second reactive moiety can be reactive with one or more moieties present in the pH-sensitive aptamer switch. In some embodiments, the monvalent linking group comprises an alkyl group having from 1 to 6 carbon atoms in its backbone.

A pH-sensitive aptamer switch for particular analyte of interest can be selected in view of a number of considerations including analyte identity, analyte concentration, and the nature of the sample in which the analyte is to be detected.

As used herein, the term “analyte” is a broad term and is used in its ordinary sense and includes, without limitation, any chemical species the presence or concentration of which is sought in material sample by the sensors and systems disclosed herein. In some cases, the analyte of interest can comprise a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or biomolecule.

For example, the analyte(s) include, but not are limited to, glucose, ethanol, insulin, water, carbon dioxide, blood oxygen, cholesterol, bilirubin, ketones, fatty acids, lipoproteins, albumin, urea, creatinine, white blood cells, red blood cells, hemoglobin, oxygenated hemoglobin, carboxyhemoglobin, organic molecules, inorganic molecules, pharmaceuticals, cytochrome, various proteins and chromophores, microcalcifications, ions, electrolytes, sodium, potassium, chloride, bicarbonate, and hormones. In one embodiment, the analyte can be drug, such as cocaine. In one embodiment, the analyte can be a neurotransmitter, such as serotonin or dopamine. In various embodiments, the analytes can be other metabolites or biomarkers of interest.

In some embodiments, the analyte of interest can comprise a small molecule (e.g., an organic molecule having a molecular weight of less than 1,000 Da, less than 800 Da, or less than 500 Da).

In some embodiments, the pH-sensitive aptamer selectively associates with the analyte of interest (e.g., in the first state at a first pH). The term “selectively associates”, as used herein when referring to a recognition element such as an aptamer, refers to a binding reaction which is determinative for the analyte of interest in a heterogeneous population of other similar compounds. Generally, the interaction is dependent upon the presence of a particular structure (e.g., an antigenic determinant or epitope) on the binding partner. By way of example, an aptamer selectively associates to its particular target when the aptamer binds to the analyte of interest but it does not bind in a significant amount to other molecules present in the sample.

In some embodiments, in its first state, the pH-sensitive aptamer can exhibit an affinity constant (Kₐ) greater than about 10⁵ M⁻¹ (e.g., greater than about 10⁶ M⁻¹, greater than about 10⁷ M⁻¹, greater than about 10⁸ M⁻¹, greater than about 10⁹ M⁻¹, greater than about 10¹⁰ M⁻¹, greater than about 10¹¹ M⁻¹, greater than about 10¹² M⁻¹, or more) with the analyte of interest.

In some embodiments, the pH-sensitive aptamer can exhibit a binding affinity for the analyte of interest in the first state (e.g., at a first pH) that is at least 10, 50, 100, 250, 500, or 1000 times greater than a binding affinity for the analyte of interest in the second state (e.g., at a second pH).

As discussed in more detail in the examples, the sensor can further include electronic circuitry configured to detect a change in an electrical property of the potentiometric sensor. For example, the sensor can include electronic circuitry configured to measure a change in current flow, a change in voltage, a change in impedance, or combinations thereof. In some embodiments, the potentiometric sensor can be inductively coupled to a signal transducer, a transmitter, or a combination thereof.

### Methods of Use

The sensors described herein can be used to rapidly and accurately detect an analyte of interest.

In certain embodiments, the sensors described herein can be used to rapidly and accurately detect an analyte of interest under physiological conditions. As used herein, the term “physiological conditions” refers to temperature, pH, ions, ionic strength, viscosity, and like biochemical parameters which exist extracellularly or intracellularly in an organism. In some embodiments, the physiological condition refers to conditions found in serum and/or blood of an organism. In some embodiments, the physiological condition refers conditions found in a cell in an organism.

Particular in vitro conditions to mimic physiological conditions can be selected by the practitioner according to conventional methods. For general guidance, the following buffered aqueous conditions can be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants. In general, in vitro conditions that mimic physiological conditions comprise 50-200 mM NaCl or KCl, pH 6.5-8.5, 20-45° C., and 0.001-10 mM divalent cation (e.g., Mg²⁺, Ca²⁺); preferably about 150 mM NaCl or KCl, pH 7.2-7.6, 5 mM divalent cation.

Methods for detecting an analyte of interest can include contacting the analyte of interest with a sensor, and measuring a change in an electrical property of the potentiometric sensor. The change in electrical property can be, for example, a change in current flow, a change in voltage, a change in impedance, or combinations thereof.

The methods described herein can be used to detect analytes in solution. In some embodiments, the analyte of interest is present in an aqueous solution.

The analyte of interest can be present in a biological sample. “Biological sample,” as used herein, refers to a sample obtained from or within a biological subject, including samples of biological tissue or fluid origin obtained in vivo or in vitro. Such samples can be, but are not limited to, bodily fluid, organs, tissues (e.g., including resected tissue), fractions and cells isolated from mammals including, humans. Biological samples also may include sections of the biological sample including tissues (e.g., sectional portions of an organ or tissue). The term “biological sample” also includes lysates, homogenates, and extracts of biological samples.

In certain embodiments, the analyte of interest is present in a bodily fluid. “Bodily fluid”, as used herein, refers to a fluid composition obtained from or located within a human or animal subject. Bodily fluids include, but are not limited to, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions, as well as mixtures containing homogenized solid material, such as feces, tissues, and biopsy samples.

The methods described herein can be used to detect an analyte of interest in vivo (i.e., the analyte of interest is contacted with the sensor in vivo). In these instances, methods for detecting an analyte of interest can include advancing or implanting a sensor into a patient, contacting the analyte of interest within the patient with the sensor, and measuring a change in an electrical property of the potentiometric sensor.

The methods described herein can be used to detect an analyte of interest ex vivo (i.e., the analyte of interest is contacted with the sensor ex vivo). The term “ex vivo,” as used herein, refers to an environment outside of a subject. Accordingly, a sample of bodily fluid collected from a subject is an ex vivo sample of bodily fluid. In these instances, methods for detecting an analyte of interest can include collecting a biological sample from a patient, contacting the analyte of interest in the biological with a sensor, and measuring a change in an electrical property of the potentiometric sensor. In certain embodiments, the ex vivo sample is a biological fluid, lysate, homogenate, or extract.

The methods described herein can be used to detect an analyte of interest in vitro (i.e., the analyte of interest is contacted with the sensor in vitro). Such methods can be used, for example, to monitor tissue cultures.

The analyte of interest can be present in an environmental sample, such as a water sample or soil leachate.

The methods can be used to determine a presence of the analyte of interest, to determine the concentration of the analyte of interest, or a combination thereof.

The sensors described herein can be integrated into devices to facilitate the detection of analytes in vivo, ex vivo, and in vitro. For example, the sensors described herein can be integrated into a variety of existing medical devices, clothing, research instruments, and vessels to provide a real-time capability for rapidly and accurately assaying the presence of one or more analytes of interest.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. A Wireless, Regeneratable Cocaine Sensing Scheme Enabled by Allosteric Regulation of pH Sensitive Aptamers

A key challenge for achieving continuous biosensing with existing technologies is the poor reusability of the bio-recognition interface due to the difficulty in the dissociation of analytes from the bio-receptors upon surface saturation. In this Example, we introduce a regeneratable biosensing scheme enabled by allosteric regulation of a re-engineered pH sensitive aptamer (in this Example an anti-cocaine aptamer). The aptamer can regain its affinity with target analytes due to proton-promoted duplex-to-triplex transition in DNA configuration followed by the release of adsorbed analytes. A Pd/PdHₓ electrode placed next to the sensor can enable the pH regulation of the local chemical environment via electrochemical reactions. Demonstration of a ‘flower-shaped’, stretchable, and inductively coupled electronic system with sensing and energy harvesting capabilities provides a promising route to designing wireless devices in bio-integrated forms. These advances have the potential for future development of electronic sensing platforms with on-chip regeneration capability for continuous, quantitative, and real-time monitoring of chemical and biological markers.

### INTRODUCTION

The allosteric regulation of triplex nucleic acid helices has offered opportunities in controlling the affinity between ligands and receptors and thus has received considerable attention as a rich “toolbox” for multiple purposes. Briefly, this strategy utilizes DNA sequence(s) capable of assembling into an inter- or intra-molecular triplex structure through Hoogsteen interactions in acidic environments. Interests in fundamental aspects of the design of aptamers and their applications in different areas (e.g., nanomachines, logic circuits, stimuli-responsive hydrogels) have motivated continued research efforts in this field. In particular, pioneering studies suggest that the pH-induced, switchable duplex-to-triplex transition can result in the disruption of the stem-loop structure of aptamers within a short time (<100 s) followed by the release of binding ligands. Despite the great success in this field, the design principles and integration schemes based on this concept for creating bio-integrated electronics are worth further study.

This Example reports an engineering solution to the challenge in continuous biosensing by using allosteric DNA-based aptamers as the sensing interface for electronic biosensors. As an example, cocaine is a highly addictive stimulant drug, and the abuse of it may cause instantaneous adverse effects on the human body, including tachycardia, hypertension, anxiety, organ damage, and immunodeficiency. Motivated by the need for sensing platforms monitoring the concentration of cocaine, this Example exploits a re-engineered anti-cocaine aptamer with a pH sensitive domain introduced to the original ligand recognition sequence to establish the proof-of-concept of regeneratable biosensors. Results suggest that an allosteric DNA functionalized surface shows pH tunable performance and can restore the sensing capability via the proper treatment of the interface in a proton-rich environment. Inspired by recent successes on electrical signal-mediated release of drugs/chemicals to form communication loops, placing a pH regulating Pd electrode close to the sensing interface can reversibly and focally control the pH value of the micro-environment, triggering the regeneration while ensuring a minimal perturbation to surrounding areas. Inductively coupling the biochemical interfaces to a custom-designed signal transducer and energy harvesting system allows for wireless sensing/pH regulation. Together, the design principles, materials selections, circuit layout, and integration scheme provide a realistic and promising route to building regeneratable biochemical sensors, which can potentially be adapted to different aptamer-mediated sensing systems. The major contribution of this work is the development of a method that enables in situ sensor regeneration under mild and biocompatible conditions together with the supporting wireless sensing/actuation schemes. The system can find potential applications for in vivo, continuous biosensing.

### Results and Discussion

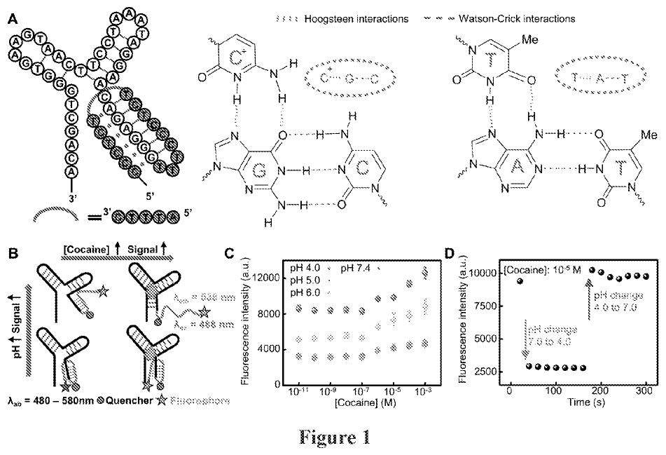

Characterization of pH sensitive, allosteric anti-cocaine aptamer. This Example uses a re-engineered DNA containing a pH sensitive domain at the 5′ end distal from the classic cocaine recognition sequence (FIG. 1(A), left). The motif includes of two self-complementary sequences separated by a loop and is capable to self-assemble with the stem of the recognition sequence into an intramolecular triplex structure through parallel Watson-Crick and Hoogsteen interactions between base pairs (i.e., CGC⁺/TAT). For a CG-rich sequence, the formation of a stable triplex is energetically more favorable in acidic environments due to the need for protonated cytosine (at N3 site) for the Hoogsteen interaction. With a decreasing pH, the self-assembly of the intramolecular triplex disrupts the stem of the cocaine-aptamer complex with a three-way junction structure, leading to the release of ligand. At higher pH values, on the other hand, the unfolding of the triplex due to lack of hydrogen bonds restores the function of the aptamer for cocaine binding.

Labeling the DNA strand with Fluorescence Resonance Energy Transfer (FRET) pairs allows for the characterization of the dynamic conformational change of the aptamer in response to varying cocaine concentrations and pH values (FIG. 1(B)). Placing the fluorophore (ATTO488) and Black Hole Quencher-1 (BHQ-1) at different locations in the sequence can enable the visualization of the reversible duplex-to-triplex transition by measuring the fluorescence emission spectrum in the wavelength range from 530 to 550 nm with an excitation wavelength of ˜488 nm. Consistent with the mechanism described in FIG. 1(B), a lower pH promotes the formation of the triplex, resulting in a decreased fluorescence intensity by bringing the fluorophore closer to the quencher, and vice versa (FIG. 1(C)). The cocaine concentration also affects the conformation by competing with the pH sensitive motif for interacting with the stem part in the cocaine recognition sequence. As a result, increasing the concentration of cocaine suppresses the transition to the triplex, leading to an enhanced fluorescence intensity with the same pH value in cases examined here (pH from 4.0 to 7.4). The transition between duplex and triplex is transient and reversible (FIG. 1(D)): adjusting the pH values (between 4.0 and 7.0) induces an instant change in the fluorescence signal which goes back upon the reverse of the pH to the original value.

To demonstrate the reversible, dynamic duplex-to-triplex transition process in cocaine solution with concentration of 10⁻⁵ M, the real-time fluorescence emission intensity (excitation and emission wavelength range: 450-490 and 500-540 nm, 5× speed) was captured by a microscope (EVOS M5000, ThermoFisher). Results suggest that the fluorescence intensity decreases immediately following a lowering in the pH value (pH 5.0) of the environment and increases when the pH goes back to neutral (pH 7.0). Repeating this process for multiple cycles confirms the reversibility of the conformational alternation. The proposed mechanism for allosteric regulation and the results consistent with previous studies serve as the foundation for the design of regeneratable electronic biosensors for cocaine in the following sections.

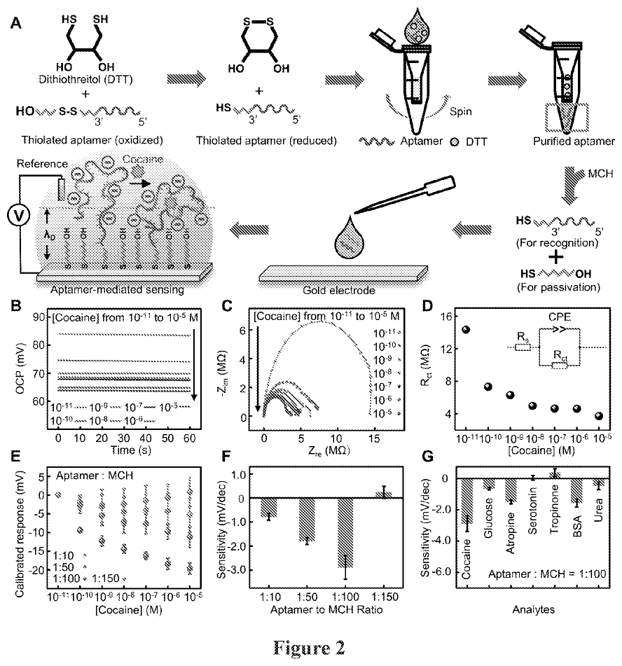

Design of anti-cocaine aptamer functionalized sensing interfaces. Modifying electrode surfaces with aptamers as the bio-recognition elements enables the generation of a quantifiable electric voltage signal that scales with the concentration of target analytes. A schematic illustration of the preparation and working principle of an aptamer functionalized cocaine sensor appears in FIG. 2(A). The interaction between target molecules and the corresponding aptamers can induce a conformational rearrangement of the single-stranded DNA with negatively charged phosphodiester backbones. Consequently, the perturbation in surface charges within the Debye length produces a measurable electrical signal readout scaling with the concentration of target analytes that can be determined by either using a transducer (e.g., field-effect transistors) or directly measuring the open circuit potential (OCP) between the sensing electrode (SE) and a reference electrode (RE). A layer of 6-Mercapto-1-hexanol (MCH) occupies the rest of the Au surface, providing passivation to block non-specific interactions. FIG. 2(B) shows the values of OCP of an aptamer functionalized commercial gold disk electrode in 1× phosphate-buffered saline (PBS) solutions with different concentrations of cocaine ranging from 10⁻¹¹ M to 10⁻⁵ M (aptamer to MCH ratio=1:100, sensitivity=−2.89±0.49 m V/dec). The monotonic decrease in OCP values with an increasing concentration of cocaine suggests the reorientation of DNA strands closer to the surface upon the binding event, bringing more negative charges into the electric double layer (EDL). The sensitivity under high ionic strength conditions is particularly advantageous for directly measuring target samples in point-of-care and/or in vivo applications. In contrast, tests in 0.1×PBS (pH=7.4) (FIG. 7) suggest a lower stability and a larger sample-to-sample variation, with an extracted sensitivity of ˜−1.13±0.16 m V/dec. This observation may be due to the reduced stability of secondary structures and binding conformation of aptamers with a decreased number of ions in the environment. FIGS. 2(C) and 2(D) show results of the electrochemical impedance spectroscopy (EIS) of the functionalized surface in different test solutions. Fitting the Nyquist plots based on a Randles circuit model shown in the inset estimates the charge transfer resistance (R_{ct}) between the redox probes ([Fe(CN)₆]^{3-/4-}) and the sensing interface (Table 1), which shows a decreasing trend upon the binding of more cocaine. It is important to note that other competing mechanisms, such as the charge of cocaine in solution and the steric repulsion between aptamers, may also affect the measured results. To this end, systematically varying the ratio of aptamer to MCH provides insight into the structure-property interrelationship. The calibrated response-concentration plots and extracted sensitivities for different sensors appear in FIGS. 2(E) and 2(F): the value of sensitivity of devices with a ratio of 1:10, 1:50, 1:100 and 1:150 corresponds to −0.79±0.14, −1.79±0.14, −2.89±0.49 and 0.23±0.25 mV/dec, respectively. The results indicate an optimized sensitivity with the ratio of 1:100. The following part of the Example exploits this ratio for sensor development.

TABLE 1
Values of Rₛ, R_{ct} and [symbol] (=P1n1) obtained by fitting the Nyquist
plots following equivalent circuit (aptamer with the pH-sensitive motif).

| Concentration (M) | Rₛ (ohm) | R_{ct} (ohm) | P1 | n1 |
|---|---|---|---|---|
| 10⁻¹¹ | 581.44 | 1.4911E07 | 5.194E−08 | 0.93598 |
| 10⁻¹⁰ | 5.7059E−12 | 7.0581E06 | 6.8716E−08 | 0.79686 |
| 10⁻⁹ | 2.531E−11 | 5.9674E06 | 7.6978E−08 | 0.76548 |
| 10⁻⁸ | 2.9221E−11 | 4.8187E06 | 7.4275E−08 | 0.80636 |
| 10⁻⁷ | 3.9725E−11 | 4.4146E06 | 7.9194E−08 | 0.78284 |
| 10⁻⁶ | 8.8495E−12 | 4.2693E06 | 7.8232E−08 | 0.79395 |
| 10⁻⁵ | 4.1198E−11 | 3.5318E06 | 7.4538E−08 | 0.82401 |

TABLE 2
Values of Rₛ, R_{ct} and [symbol] (=P1n1) obtained
by fitting the Nyquist plots following equivalent
circuit (aptamer without the pH-sensitive motif).

| Concentration (M) | Rₛ (ohm) | R_{ct} (ohm) | P1 | n1 |
|---|---|---|---|---|
| 10⁻¹¹ | 608.03 | 5.1371E+06 | 3.9001E−08 | 0.94280 |
| 10⁻¹⁰ | 574.45 | 3.9036E+06 | 4.0714E−08 | 0.93831 |
| 10⁻⁹ | 566.8 | 3.7846E+06 | 4.0946E−08 | 0.93808 |
| 10⁻⁸ | 571.16 | 3.6579E+06 | 4.1452E−08 | 0.9368 |
| 10⁻⁷ | 543.82 | 3.4655E+06 | 3.8762E−08 | 0.93917 |
| 10⁻⁶ | 482.7 | 1.8580E+06 | 4.4544E−08 | 0.92604 |
| 10⁻⁵ | 514.08 | 1.7525E+06 | 4.2448E−08 | 0.92943 |

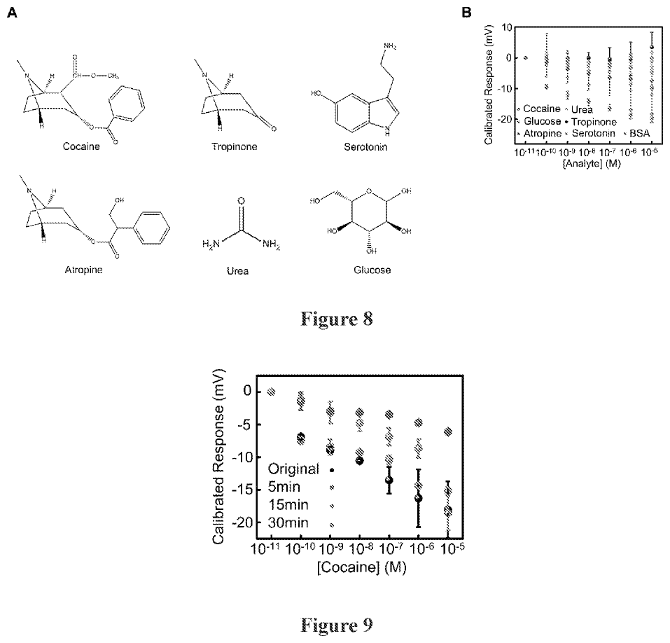

Measuring responses to non-specific binding chemicals evaluates the selectivity of this sensing platform. Biomolecules having a considerable concentration in bodily fluids (e.g., glucose, urea, lactate, BSA) and a similar chemical structure/molecular weight (e.g., atropine, tropinone) are potential interferents of interest. Additionally, a recent study shows that cocaine can increase endogenous serotonin in the ventral pallidum, and therefore, this study also investigates the cross-sensitivity for potential applications in the future. The chemical structures of the analytes used here, and the corresponding response-concentration plots appear in FIG. 8, with extracted values of sensitivity shown in FIG. 2(G). A sensitivity of −2.89±0.49 m V/dec for cocaine in contrast to −0.66±0.07 m V/dec for glucose, −1.51±0.11 m V/dec for atropine (due to the structural similarity), 0.04±0.15 m V/dec for serotonin, 0.37±0.24 m V/dec for tropinone, −1.57±0.26 m V/dec for BSA (due to large molecular weight) and −0.47±0.25 m V/dec for urea suggests a specificity. The mechanism underlying the response for glucose, urea, lactate, BSA and serotonin might be nonspecific interactions between analytes and the sensor surfaces, whereas that for atropine and tropinone might originate from their interactions with aptamers due to the structural similarity to cocaine. Further optimizations are possible via the proper passivation to minimize nonspecific interaction and adsorption on both the SE and RE.

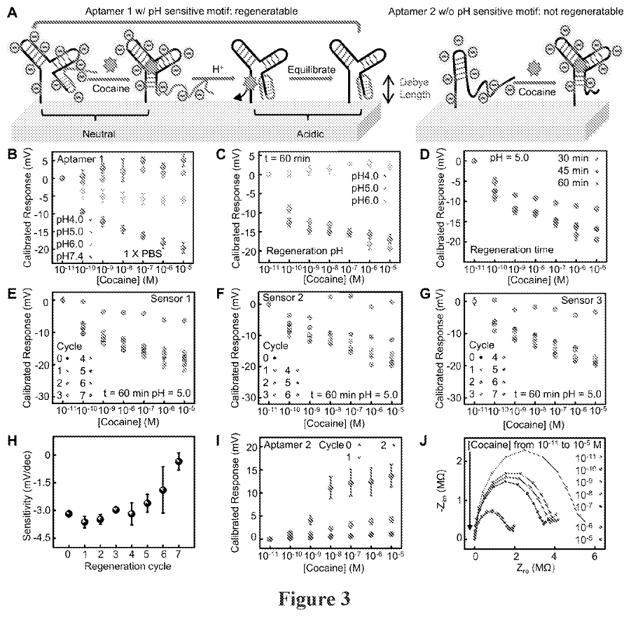

Regeneration of anti-cocaine sensors enabled by pH-induced allostery. As discussed in the preceding section, the addition of the pH sensitive motif at the 5′ end of the aptamer (denoted as Aptamer 1) can enable the duplex-to-triplex transition in the acidic environment, resulting in the disruption of the stem-loop structure followed by the release of binding ligands. Increasing the pH value of the environment will reverse the conformation by forming duplex again and thereby recover the function of the aptamers for capturing target ligands. FIG. 3(A) shows the schematic illustration of the regeneration process enabled by allostery upon pH regulation. On the other hand, the DNA strand without the pH sensitive motif (denoted as Aptamer 2) lacks the capability of forming triplex, and thereby is expected to be insensitive to pH-induced regeneration. FIG. 3(B) shows responses of Au electrodes functionalized with Aptamer 1 to cocaine solutions (1×PBS) with varying concentrations and different pH values (4.0, 5.0, 6.0, and 7.4). The results suggest a decreasing trend in sensitivity as the environment becomes more acidic which is consistent with the mechanism stated above. Interestingly, studies under the same test conditions but with 0.1×PBS cannot provide stable, monotonic responses in any cases (FIG. 7). The observation further supports that the ionic coordination may influence the loop topology of the DNA strand and play an important role in stabilizing the anti-cocaine aptamers used in this work.

Treating cocaine adsorbed devices (after completing measurement in 10⁻⁵ M cocaine solution samples) with acidic solutions allows for regeneration based on the duplex-to-triplex transition. Systematic studies investigate effects of key parameters of experimental conditions including pH value, treatment time, and agitation. To ensure a similar starting point, only devices showing an initial sensitivity of >2.5 m V/dec (absolute value) are selected for the regeneration study described below. FIG. 3(C) presents the calibrated response-concentration curves of sensors after regeneration in solutions with pH values of 4.0, 5.0, and 6.0 for 60 min. The results show that with a pH value of 5.0 or less, the sensors can restore most of the function, regaining a sensitivity of ˜−3.2 m V/dec on average. When the pH value increases to 6.0, however, used devices do not show observable sensitivity after treatment, indicative of an insufficient level of protonation of the N3 in cytosine for completely transitioning the duplex stem into triplex containing CGC⁺ for efficient ligand dissociation. FIG. 3(D) presents responses of regenerated devices after immersion in solution with a pH of 5.0 for 30, 45, and 60 min. The results demonstrate a trend that increases the treatment time can promote the restoration of sensitivity, which might correspond to the time needed for the ligand to disrupt noncovalent interactions, dissociate from aptamers, and diffuse away into the bulk solution. Systematic studies with agitation (speed: 150 revolutions per minute (rpm)) show a similar trend and a shorter time (˜15 min) for full function restoration (FIG. 9), suggesting the feasibility of achieving rapid refreshment of sensing interfaces within dynamic biosystems having continuous blood flow and tissue movement. Tests during repetitively applied sensing and regeneration cycles on three individual devices appear in FIG. 3(E) to 3 (G), and FIG. 3(H) summarizes the average value of sensitivity as a function of the regeneration cycle. Generally, an observable decrease in sensitivity starts to appear during the 5th regeneration cycle, and the value drops to near 0 during the 7^{th} cycle, which could be attributed to the degradation of surface functional groups over time in the liquid environment. The results presented here suggest the potential of using the regeneratable sensor system enabled by DNA allostery for real-time monitoring which can provide status updates on an hourly basis.

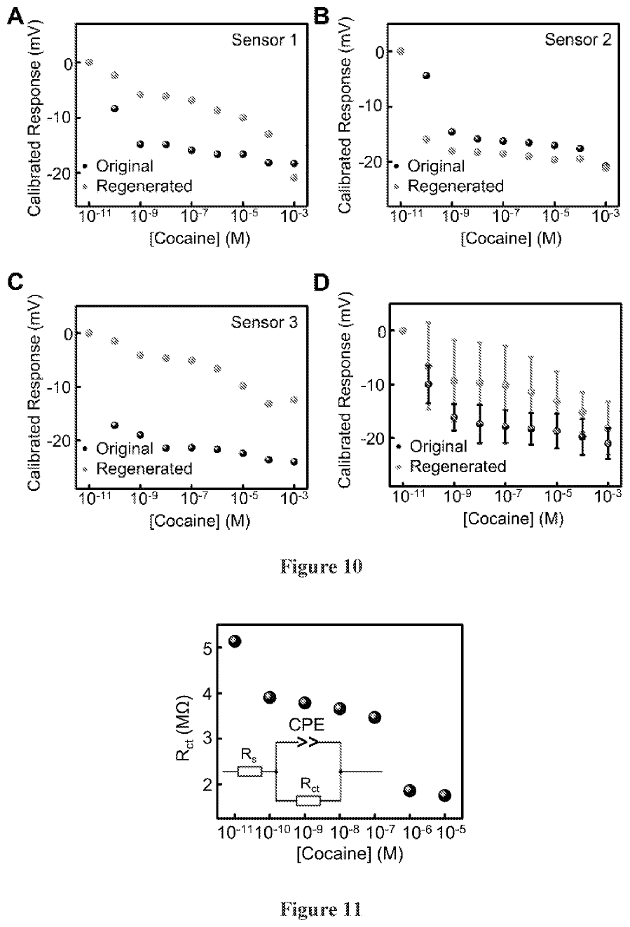

Another set of experiments explores the regeneration performance of the sensing interface after being saturated in a higher concentration cocaine solution (up to 10⁻³ M). As shown in FIG. 10, the results suggest that a higher concentration can affect the quality of the regenerated sensors: while the calibration plot after regeneration (pH=5.0, 60 min) does show a similar trend of decrease in OCP values with an increasing cocaine concentration, the extracted sensitivity (−1.79±0.39 m V/dec) is lower than the original one (−2.66±0.78 m V/dec), and the variation among different samples also increases. Two reasons might account for this observation: (1) A highly saturated sensor surface requires a longer regeneration time for the complete dissociation of adsorbed analytes; and (2) the as-purchased cocaine solution used for this study exploits acetonitrile as the solvent, and the higher amount of the organic solvent in a less diluted test solution (10⁻³ M) may cause the degradation of aptamers during the test. To demonstrate the importance of the pH sensitive motif, control experiments with Aptamer 2 having the same anti-cocaine DNA sequence but without the triplex-forming motif serve as a comparison. The response-concentration curve under the pH neutral condition shows the opposite trend as to that of Aptamer 1 with a positive value of sensitivity (2.3 m V/dec). A possible reason accounting for this observation is that Aptamer 2 may go through an opposite conformational change upon the binding of cocaine by reorienting away from the surface which consequently decreases the number of negative surface charges (as illustrated in FIG. 3(A)). In the meantime, the positive charges cocaine molecules carry may also contribute to the increase in surface potential. Interestingly, fitting values (Table 2, FIG. 11) of Nyquist plots (FIG. 3(J)) demonstrate the same trend of monotonic decrease as observed for Aptamer 1, consistent with a previous study on EIS sensors for cocaine. The mechanism underlying the response is worth further investigation, which could be due to a combined effect of changes in surface potential, charges of adsorbed analytes, and steric hindrance leading to a decreased electrostatic repulsion of redox probes. Due to the lack of pH sensitivity of Aptamer 2, acid-treated sensors (pH=5.0, treatment time=60 min) show a very limited capability of regeneration, with an almost negligible sensitivity after the first acid-treatment cycle.

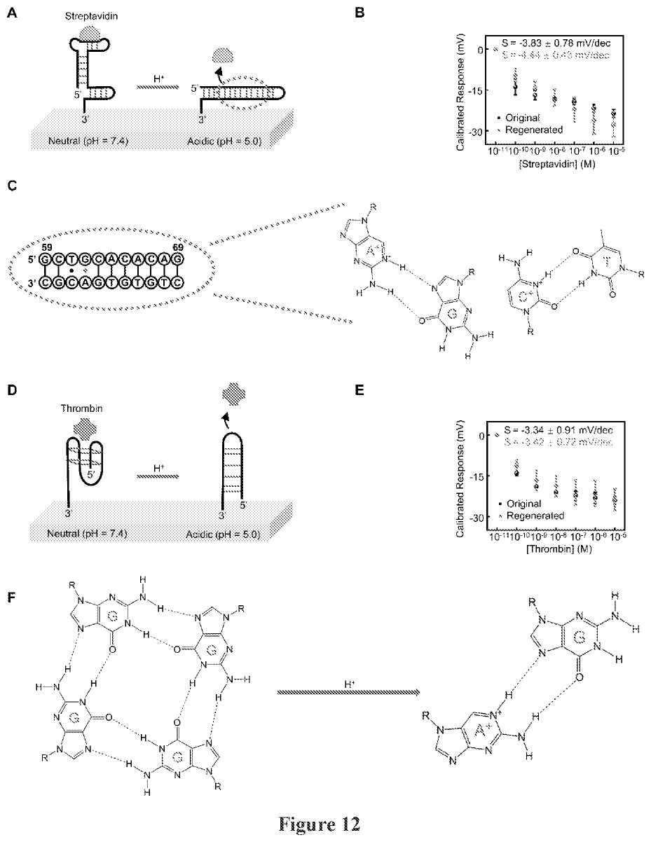

In addition to the example of regeneratable cocaine sensors, it is important to explore the versatility of this technology to other biomolecules. FIG. 12 shows the regeneration performance of sensors functionalized with pH sensitive anti-streptavidin and anti-thrombin aptamers and the underlying mechanism based on the allosteric regulation of the DNA sequences: at a neutral pH condition, both aptamers are in a folded conformation allowing for the binding of streptavidin and thrombin molecules with a high affinity. Following a very similar working principle to that of the anti-cocaine aptamers, the addition of extra protons in an acidic environment result in a mismatch between the base pair G and A, leading to a disruption of the original conformations and accordingly, the release of adsorbed analytes for surface regeneration. In both cases, the devices can fully restore the sensitivity after an acid treatment (pH=5.0) for 60 min (for streptavidin: −3.83±0.78 m V/dec (before) vs. −4.44±0.43 m V/dec (after); for thrombin: −3.34±0.91 m V/dec (before) vs. −3.42±0.72 m V/dec (after). The results on alternative biomolecules in addition to cocaine suggest that the versatility of this sensing scheme based on allosteric DNAs.

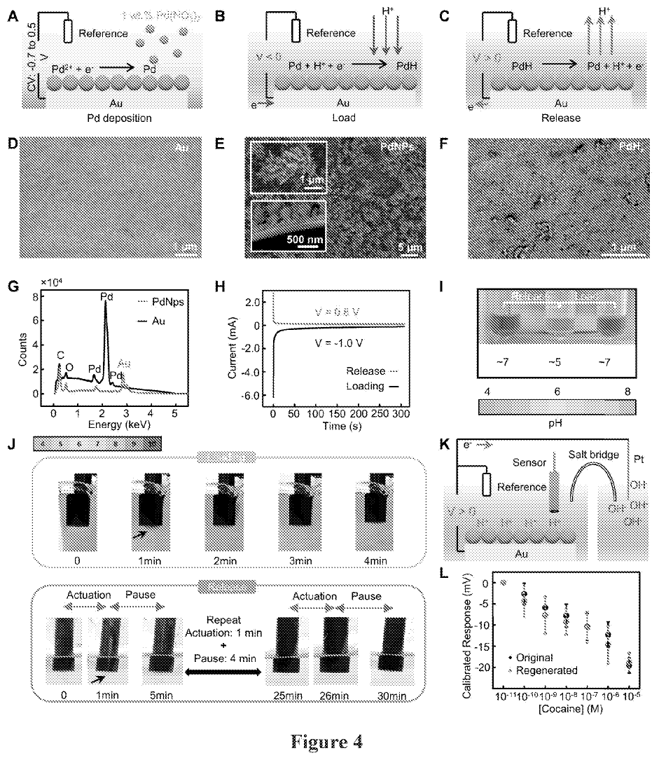

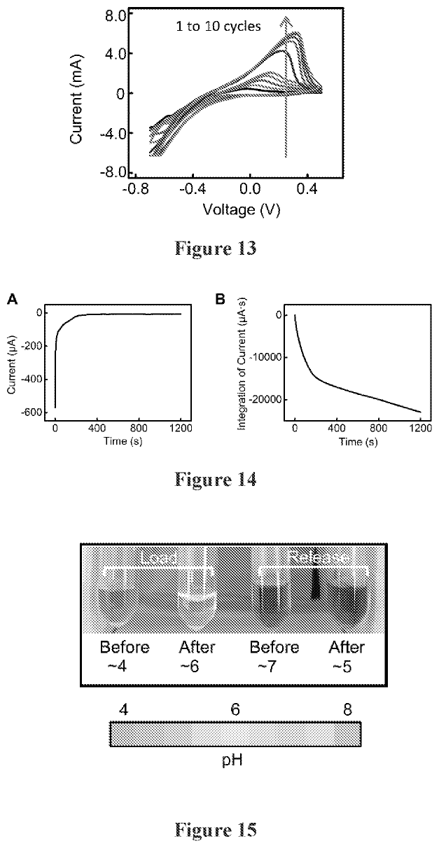

Local pH regulation with Pd/PdHₓ electrodes via electrochemical actuation. To circumvent the challenge of integrating chemical reagents within the sensor chip for regeneration, the Example presents a solution by introducing a palladium (Pd)-based bioprotonic pH regulating electrode that can control the pH value of local environments through electrochemical actuation. Unlike most metals which are good contact for electrons but poor for protons, Pd has a strong affinity to hydrogen and thereby can support the loading/release of protons via the reversible electrochemical reaction: H⁺+e⁻↔H. The formation of palladium hydride (PdHₓ) stores hydrogen, and oxidizing the resulting system can convert hydrogens back to protons again upon the application of a positive voltage, lowering the pH values of the local chemical environment. Schematic illustrations of the fabrication process and working principles for pH control appear in FIG. 4(A) to 4(C). Electrochemical deposition using cyclic voltammetry forms Pd nanoparticles (PdNps) on the surface of an Au electrode (FIG. 13). Scanning electron microscope (SEM) images of Au surfaces before/after deposition appear in FIGS. 4(D) and 4(E), inset, top. The cross-sectional SEM image (FIG. 4(E), inset, bottom) of the electrochemically deposited PdNps layer (10 cycles) suggests a thickness of ˜300 nm. The surface becomes smoother after the loading of protons as hydrogen atoms diffuse into the interstitial lattice of Pd (FIG. 4(F)). FIG. 4(G) presents the energy-dispersive X-ray spectroscopy (EDS) analysis of Au surfaces before and after the electrodeposition, confirming the successful deposition of PdNps on the electrode: the atomic percentage of Au on the surface decreases from 23.79% to 0.71% while Pd increases from 0.04% to 27.13%, respectively (Table 3 and 4).

TABLE 3
EDS elemental analysis table of Au film before PdNps deposition.

| Element | Weight % | Atomic % |
|---|---|---|
| C | 2.55 | 12.47 |
| Si | 0.49 | 1.02 |
| Au | 79.81 | 23.79 |
| Pd | 0.07 | 0.04 |
| O | 17.08 | 62.69 |

TABLE 4
EDS elemental analysis table of Au film with PdNps.

| Element | Weight % | Atomic % |
|---|---|---|
| C | 3.35 | 11.62 |
| Si | 2.04 | 3.03 |
| Au | 3.35 | 0.71 |
| Pd | 69.20 | 27.13 |
| O | 22.06 | 57.50 |

Electrochemically loading the PdNp-decorated electrode with protons in an acidic environment followed by release tests in pH neutral solution examines the function of the pH regulator: soaking the electrode in 500 mM NaCl solution (with 0.1 mM H₂SO₄) at pH 4.0 with an applied voltage of −1.0 V for 300 s allows for the adsorption and reduction of H⁺ to form PdHₓ. FIG. 14 shows the current change for an as-prepared electrode (diameter: 2 mm) over time during the loading process and results of integrate of current over time. Based on the results, the calculated total amount of proton capacity of the electrode is ˜2.39×10⁻⁷ mol. After that, immersing the electrode loaded with hydrogen atoms in 500 mM NaCl solution at pH 7.0 with an applied voltage of 0.8 V leads to the oxidation and release of protons (FIG. 4(H)). Collecting solutions (˜400 μL) in the surrounding environment near the electrode during the process and adding a universal pH indicator (Merck, a mixture of thymol blue, methyl red, bromothymol blue, and phenolphthalein, pH transition range: 4.0-10.0) qualitatively visualize the change in pH values. Please note that the color difference in FIG. 15 is due to the use of different solution samples, that is, the release test on the right does not start with the resulting solution after loading. To better demonstrate the reusability of the electrodes, FIG. 4(I) shows the reversible color change of solutions collected near the working electrode during the repetitive loading/release process, which further supports that the pH regulating electrode is reusable.

The actual amount of solution going through the pH change depends on the dimension of the electrode (diameter: 3 cm for 4(I) and 2 mm for 4(J)). The purpose of using FIG. 4(J) is to show that the loading and release processes only modify the local chemical environment near the electrode surface (as indicated by the arrows) while that for the rest of the bulk solution can remain unchanged. Taking sequential photographs during the loading/release processes with the pH indicator evaluates the concentration gradient of protons at the sensor-solution interface as a function of time by capturing the color change (FIG. 4(J)). The loading increases the pH value of the environment, and the system reaches equilibrium within ˜5 min. As discussed earlier, while the conformational switch of aptamers usually takes place rapidly in response to pH regulation, the time needed for the dissociation of substrates from the aptamers represents the bottleneck for sensor regeneration. Consequently, it is important to maintain an acidic chemical environment over a prolonged period (i.e., at least 10-20 min, according to results in FIG. 9). Since a sudden release of all protons in the electrode may create an overconcentrated acid environment, the study exploits a cyclic electrochemical actuation protocol with an application of voltage for 1 min followed by a pause for 4 min to fully make use of the loaded protons and to provide a relatively mild condition for potential applications in biosystems. As shown in the sequential photographs, the concentration gradient can remain at the sensor surface after 6 cycles (30 min). Placing a cocaine-adsorbed Au electrode close to an as-prepared pH regulating electrode and conducting the cyclic actuation validate the regeneration scheme (FIG. 4(K)). To ensure high efficiency of regeneration, the study separates the working and counter electrodes using a salt bridge, as the reduction reaction occurs simultaneously at the counter electrode, resulting in an increase in local pH values. FIG. 4(L) shows the response plots of the original and regenerated sensors with a sensitivity of −3.26 and −3.13 mv/dec, respectively. The negligible difference suggests the feasibility of using this scheme for restoring the sensing capability of allosteric DNA-functionalized sensors.

Design and characterization of bio-integrated, wireless electronic systems. The platform combining the allosteric aptamer functionalized interface and the pH regulator offers a route to developing reusable biosensors. In addition to the biochemical interfaces, for health monitoring purposes, it is also important to design matching coupling strategies that enable the transmission of sensing data and powering of the pH regulating electrode, ideally, in a wireless manner.

To address this issue, the Example presents a regeneratable “flower-shaped” sensor prototype for wireless sensing and electrochemical actuation. FIG. 5(A), left shows the schematic illustration of a stretchable “flower-shaped” device based on the circuit model. The device consists of three key functional parts: (1) “petals”: a biochemical interface with a pair of SE and RE surrounded by a PdNps decorated metal trace, (2) “leaf”: an inductive coupling unit with one coil for data transmission and another coil for RF energy harvesting and electrochemical actuation, and (3) “stem”: stretchable wires connecting part (1) and (2). Patterning the wires into serpentine structures enables the distribution of mechanical strains and protection of functional parts from deformation when integrated with biotissues. This modularized device also allows for the physically separated placement of part (1) and (2) to provide seamless integration with target biotissues/biofluids and efficient, stable coupling with external electronics. FIG. 5(A), right shows envisioned applications based on the sensor prototype, including using custom-designed devices as wearable, implantable, and point-of-care systems. The benefits for having a cocaine sensor wireless and stretchable is to enable the transmission of sensing data and powering of the pH regulating electrode in a bio-integrated manner for continuous monitoring of drug levels within the human body. For wearable electronics, this sensing platform can be worn on different locations such as forehead, arm, back and so on whereas the functional sensing/actuation part can extend to places of interest with biofluids. FIG. 5(B) demonstrates photographs of the “flower-shaped” device in flat and bent configurations highlighting its flexibility.

The equivalent circuit diagrams for the sensor and actuator are in FIG. 5(C). For the biochemical sensing circuit, the surface potential change caused by analyte-aptamer interaction serves as a reverse bias for a pair of varactor diodes, which modifies the thickness of the depletion region in the p-n junction in the diodes. The modification leads to a change in capacitance in an inductor-capacitor resonance circuit and shifts the resonance frequency according to the following equation:

$${f}_{s} = \frac{1}{2 π \sqrt{LC}}$$

where L and C are the inductance and capacitance of the circuit, respectively. Aligning the coupling unit with a readout coil connected with a vector network analyzer and sweeping the frequency record the input return loss (S11), and fitting the curve determines fₛ for the quantitative analysis of surface potential change scaling with the change in concentration of cocaine. On the other hand, the pH regulating circuit consists of a Pd-coated metal trace surrounding the sensing site and an inductive coupling unit which can wirelessly capture RF power in the range of 13-14 MHz (transmitting frequency=13.3 MHz, which falls into this range to ensure maximized energy harvesting efficiency) delivered through a vertically aligned transmission antenna. A full-wave bridge rectifier (for AC to DC conversion), a smoothing capacitor (for evening out fluctuations) and a Zener diode regulator (490 ohm, for voltage stabilization) then convert the harvested energy into a DC voltage (˜1.1 V) that can support the release of pre-loaded protons for localized pH modulation. Placing the counter electrode away from the sensing area minimizes the effect of the reduction reaction increasing the local pH around it which may suppress the efficient aptamer regeneration. Details about the design, operation, and characterization appear below.

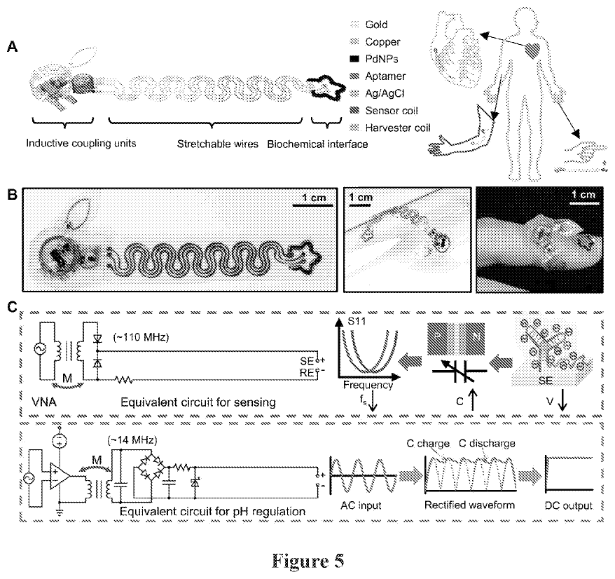

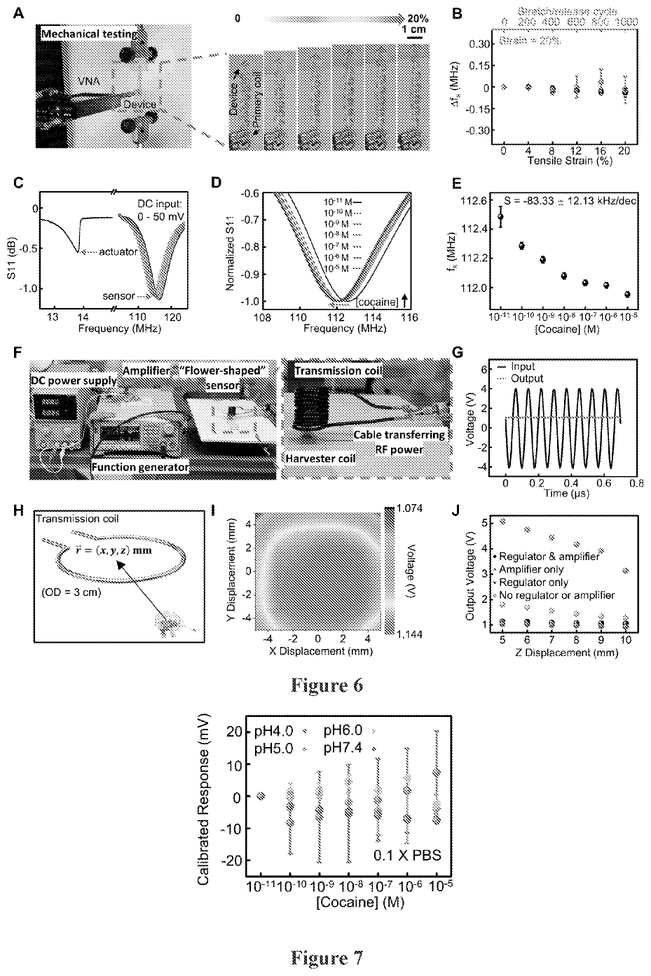

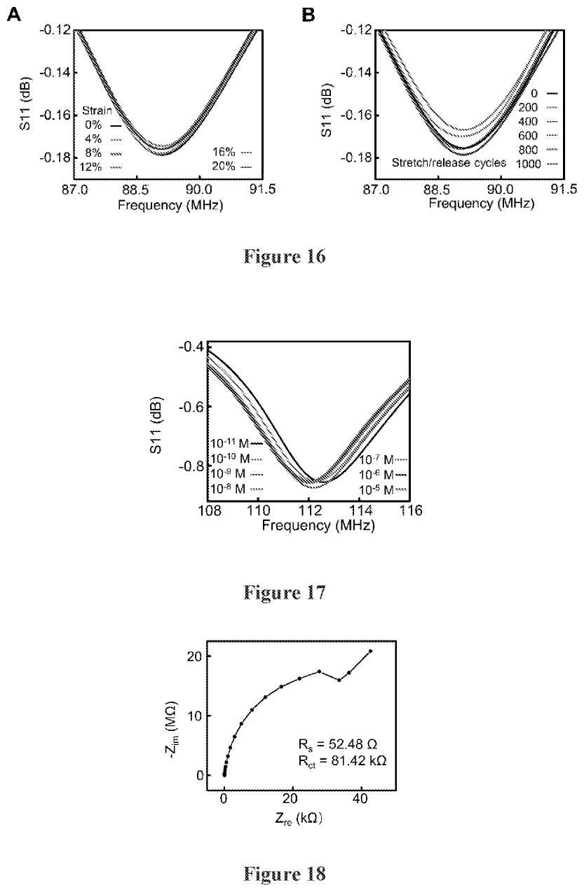

Preparing the metal traces for the three key parts using a simple “cut-and-paste” method followed by soldering electronic components and connecting them with silver epoxy yields the resulting flexible electronic system. The low modulus and high elasticity of the device can significantly decrease the probability of mechanical failure when serving as bio-integrated electronics. Cyclic stretching tests evaluate the mechanical robustness of the system. FIG. 6(A) shows photographs of a test device subject to a strain of up to ˜20% which encompasses the elastic stretchability of the epidermis (˜15%). Here incorporating a damping resistor (10 Kohm) in series with the SE and RE isolates the DC circuitry from the coupling unit and minimizes variations in fₛ due to changes in parasitic inductance/capacitance resulted from mechanical deformation. FIGS. 6(B) and 16 show fₛ with varied tensile strains and before/after a different number (0-1000) of stretch/release cycles. The value of fₛ remains largely unchanged throughout the test with little dependence on the tensile strain, highlighting the potential of the system serving as bio-integrated electronics on non-flat surfaces with external strains.

A rational design of the wireless sensor and actuator separates their operational frequencies for individual functions. Additionally, increasing the number of coils turns of the coupling unit for the actuator leads to a larger magnetic flux serving as the power supply for pH modulation. Specifically, this study uses a 5-turn (diameter: 3 mm) and a 20-turn (diameter: 5 mm) coil for the sensor and actuator, and the resonance curves appear in FIG. 6(C) with fₛ of ˜115 MHz and 14 MHz, respectively. Applying a reverse bias voltage ranging from 0-30 mV across the SE and RE and recording the resonance frequency with an external antenna connected to a portable vector network analyzer (NanoVNA) placed on the top examine the performance of the circuit in transmitting static DC input simulating surface potentials associated with the potentiometric sensor design. fₛ of the coupling unit shifts to larger values with an increasing bias based on the voltage frequency modulation working principle described in the preceding section, yielding a voltage sensitivity of 0.042 MHz/mV. Following this principle, connecting the functionalized electrode pairs to the wireless circuit (SE with cathode, and RE with anode) yields a sensing system capable of detecting concentration of cocaine based on the potentiometric sensing mechanism. Exposing the sensor part to cocaine solutions with varying concentrations results in a shift in fₛ due to modification in surface potential (FIGS. 6(D) and 17). FIG. 6(E) shows the extracted fₛ as a function of cocaine concentration, with a sensitivity of −83.33±12.13 kHz/dec. The above results demonstrate that the platform possesses sufficient voltage sensitivity for wireless chemical sensing.

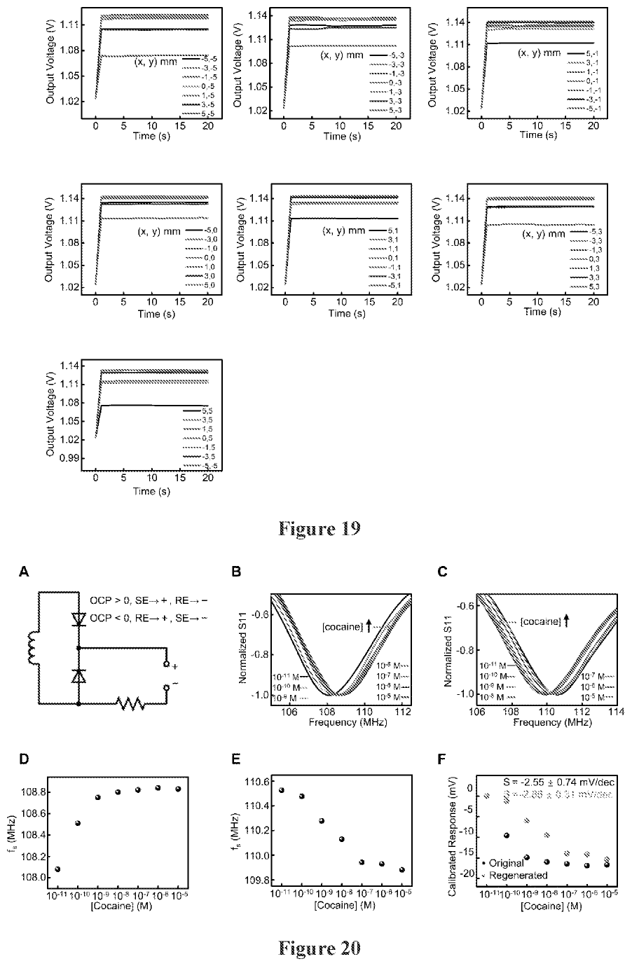

Beyond the sensing capability, magnetic resonance coupling serves a simple, straightforward, and competing technique for powering wireless bioelectronics due to the resistance to environmental interference. Additionally, separating the energy harvester from the PdNp-functionalized interface matches the design of the modularized sensor system. The “two-part” design provides opportunities for building bio-integrated electronics with advanced functions and improved stability in performance. To evaluate the performance in power transfer, systematic studies investigate the effect of relative position change between the transmitter (OD=3 cm) (connected to a function generator and a power amplifier) and receiver (connected to an electrochemical workstation) in three directions along a vector (starting and end points: the center of the harvester and transmission coils, respectively) (FIG. 6(H)). The setup of the experiment appears in FIG. 6(F): a function generator (V_{AC, sin}=˜10/pp, transmitting frequency=13.3 MHz) supplies a continuous RF energy followed by power amplification. The inductive coupling between the transmitter and receiver delivers the RF electrical power to the electrochemical actuation system followed by waveform rectification and voltage regulation. FIG. 6(G) illustrates the amplified waveform wirelessly supplied to the transmitter coil (measured across the output ports of the cable after power amplification) and the harvested DC voltage measured across a load resistor (10 Kohm, determined based on the EIS spectrum of PBS solution, FIG. 18) connected to the interface for pH modulation. The frequency separation and the lateral displacement between the coupling units for the sensor and actuator prevent unexpected activation of non-target components. FIG. 6(I) shows a mapping of the output voltage corresponding to relative position displacement in (x, y) from (˜5,−5) mm to (5, 5) mm, with z=5 mm (raw data in FIG. 19). The output voltage remains highly stable throughout the test ranging from 1.074 to 1.144 V, which meets the requirement of Pd/PdHₓ electrodes for the oxidation reaction. Similarly, FIG. 6(J) shows the results with (x, y) fixed at (0, 0) and a varying distance along the z-axis. For the two control groups without the voltage regulator (red and orange), the difference in voltage output at the same position (>two times) suggests the importance of the power amplifier to ensure a sufficient supply of energy for the electrochemical actuation. The results without the voltage regulator also demonstrate a considerable variation in output voltage signals throughout the measurement range. On the contrary, adding the voltage regulator minimizes displacement-associated variation in voltage, providing an enhanced stability for controlled electrochemical actuation. Overall, the characterizations of wireless sensing and RF energy harvesting suggest the potential application of the inductively coupled electronic system in facilitating closed-loop biomarker detection and in vivo regeneration when combined with a properly designed sensing interface and pH regulating materials.

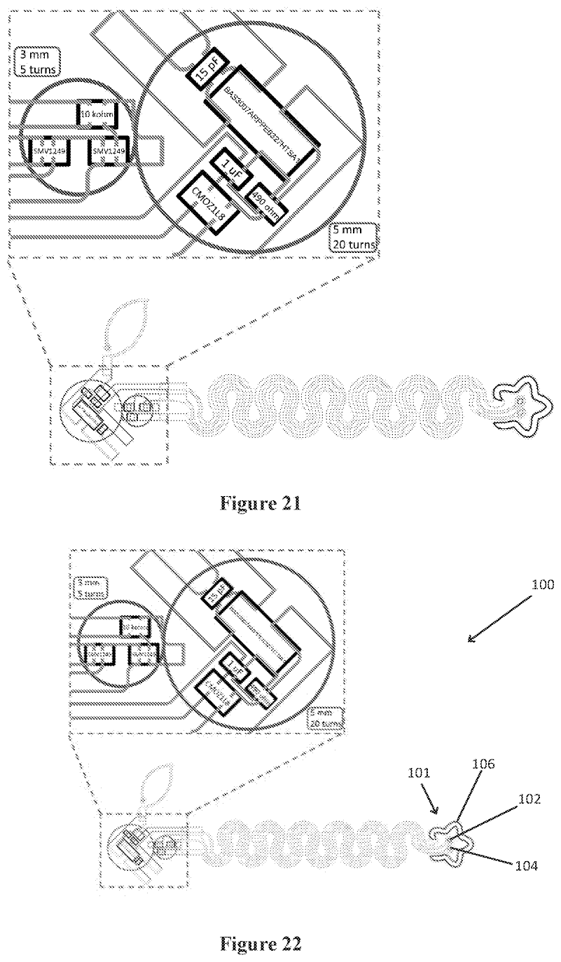

FIG. 20 shows the regeneration performance of a packaged “flower-shaped” stretchable wireless device with on-chip serpentine wires, aptamers and PdNps. Details about the connection between the inductive coupling unit and the SE/RE pairs based on their polarity appear in FIG. 20(A). FIGS. 20(B) and (C) present the resonance curves showing the shifts in fₛ due to the changes in the cocaine concentration before and after the regeneration process enabled by the pH regulator (following the actuation protocols in FIG. 4(J)). FIGS. 20(D) and (E) show the extracted fₛ as a function of cocaine concentration, and FIG. 20(F) presents the calibrated OCP response calculated based on data in FIGS. 20(D) and (E) (before: −2.55±0.74 m V/dec, after: −2.86±0.31 m V/dec). The results demonstrate the functionality and reusability of the packaged system, further highlighting the mechanical stability and energy harvesting capability of the stretchable device.

### CONCLUSIONS

In summary, the results presented in this study describe an interface design strategy with allosteric DNA as bioreceptors and an associated integration scheme for building regeneratable biochemical sensors. The resulting system combines LC resonance circuits, stretchable design, Pd-based bioprotonic pH regulator, and anti-cocaine aptamer-based biosensing interface. As a case study, a re-engineered anti-cocaine aptamer with a pH sensitive domain can regain its affinity with target analytes due to proton-promoted duplex-to-triplex transition in DNA configuration followed by the release of adsorbed ligands. Sensitivity studies using aptamer functionalized Au surfaces suggest the capability of this platform in measuring cocaine concentrations across a wide concentration range (10⁻¹¹ to 10⁻⁵ M) via a potentiometric sensing strategy. Systematic studies verify the reusability of this anti-cocaine aptamer upon proper treatment in acidic environments. Additionally, placing a Pd/PdHₓ electrode and releasing protons via electrochemical reactions allow for the pH regulation of the local chemical environment. Demonstration of a ‘flower-shaped’, stretchable, and inductively coupled electronic systems with sensing and energy harvesting capabilities provides a promising route to designing wireless devices in bio-integrated forms customizable for multiple application scenarios. Although the current study focuses on cocaine sensing as a proof-of-concept, when combined with other types of SELEX-enabled allosteric DNAs, the resulting system can readily extend to alternative biomarkers. Overall, this study sets the stage for developing promising engineering tools for continuous monitoring of biological markers through seamless and stable integration with target biosystems.

### Materials and Methods

Materials and reagents used for this study. PBS, Tris-EDTA buffer solution (TE), Cocaine solution (C₁₇H₂₁NO₄, 1.0 mg/mL in acetonitrile, ≥99%), Glucose (D-glucose, ≥99%), Atropine (C₁₇H₂₃NO₃, 1.0 mg/mL in acetonitrile), 5-Hydroxytryptamine,3-(2-Aminoethyl)-5-hydroxyindole, 5-HT (Serotonin hydrochloride), Streptavidin, Thrombin (Citrate-Free, Human serum), Tropinone (99%), Bovine serum albumin (BSA), Urea, MCH, DL-Dithiothreitol (DTT, ≥99%), Hydrochloric acid (HCl), Palladium (II) nitrate (Pd(NO₃)₂) solution (99.999%, 10 WT), universal indicator solution (pH 4.0 to 10.0), Potassium hexacyanoferrate (III) (K₃[Fe(CN)₆]), Potassium hexacyanoferrate (II) trihydrate (K₄[Fe(CN)₆]·3H₂O) were purchased from Sigma-Aldrich. Single stranded DNA aptamers were synthesized by and purchased from Integrated DNA Technologies and Biosearch Technologies Inc.

Fluorescence intensity characterization. The anti-cocaine aptamer sequence with the pH sensitive motif, fluorophore (ATTO488) and quencher (BHQ-1) was synthesized by Biosearch Technologies Inc (5′-(ATTO488) CCC TCT ATT TCT CTC CCT TT (BHQ-1) GGG AGA CAA GGA AAA TCC TTC AAT GAA GTG GGT CGA CA-3′; SEQ. ID 1). Dissolving the aptamers in PBS solution (100 μM) with different pH (4.0, 5.0, 6.0 and 7.4) and mixing the resulting systems with 2×10⁻¹¹ M cocaine solution in 1:1 ratio formed a series of test solutions. Injecting the test solutions into a 96-well plate finished the preparation of samples. The characterization of fluorescence intensity took place with a SpectraMax M5 Plate Reader at 25° C. The study used an excitation wavelength of ˜488 nm and measured the emission spectrum in the wavelength ranging from 530 to 550 nm. Gradually adding cocaine solution (from 10⁻¹¹ to 10⁻³ M) into the wells enabled the characterization of fluorescence emission of the pH sensitive aptamer in the presence of the substrate with varying concentrations.

Preparation of aptamer-modified Au electrode surfaces. Thiolated anti-cocaine aptamers (with pH sensitive motif: 5′-CCC TCT ATT TCT CTC CCT TTG GGA GAC AAG GAA AAT CCT TCA ATG AAG TGG GTC GAC A/3Thio-MC3-D/-3′ (SEQ. ID 2); without pH sensitive motif: 5′-GGG AGA CAA GGA AAA TCC TTC AAT GAA GTG GGT CGA CA/3Thio-MC3-D/-3′ (SEQ. ID) 3)), thiolated anti-thrombin aptamers (5′-GGT TGG TGT GGT TGG CTC TAA AAA AAA AAA AAA A/3Thio-MC3-D/-3′ (SEQ. ID 4)) and thiolated anti-streptavidin aptamers (5′-ATA CCA GCT TAT TCA ATT ATT GAC CGC TGT GTG ACG CAA CAC TCA ATT CTT GGA TCT CGC TGC ACA CAG ATA GTA AGT GCA ATC T/3Thio-MC3-D/-3′ (SEQ. ID 5)) were synthesized by Integrated DNA Technologies. Dissolving as-purchased aptamers in 1×TE solution with 10 mM DTT reduced the disulfide bonds and yielded a solution of aptamers with —SH groups at the 3′ end (concentration: 100 μM). Centrifuging the resulting solution in a MySpin12 (Thermo Fisher Scientific) at 2038 Relative Centrifugal Force (RCF) (2600 rpm) for 4 min removed additional DTT. Mixing the purified aptamer solution with 10 mM MCH solution in TE buffer (v/v ratio: 1:1) formed the coating solution for functionalizing Au surfaces. Heating the mixture in water bath at 95° C. for 5 min converted the DNA strands in solution to fully extended conformation, and a subsequent rapid cooling step in ice bath for 15 min stabilized the resulting structure. Drop-casting the solution on an Au electrode surface (commercial gold disk electrode or thin-film Au deposited by electron-beam evaporation) and drying the system overnight at room temperature completed the immobilization of aptamers by forming Au—S bonds at the 3′ end, with MCH serving as the passivation layer blocking the rest of the Au surface. Storing functionalized electrodes in 1×PBS solution at 5° C. retained the activity of aptamers for use over an extended period.

Preparation of cocaine solutions. Diluting cocaine acetonitrile solution (1.0 mg/mL) with 1×PBS (pH 7.4) formed a set of test solutions with varying concentrations ranging from 10⁻¹¹ to 10⁻⁴ M. Similarly, dissolving atropine, glucose, and serotonin in 1×PBS yielded corresponding test solutions. For the study on the effect of pH values, adding 1 M HCl to cocaine solutions modulated pH values of the systems to 4.0, 5.0 and 6.0. For the study on the effect of ionic strength, diluting the systems with deionized (DI) water formed test solutions in 0.1×PBS.

Electrical characterization of aptamer functionalized Au electrode. Before each test, incubating the SE in the target solution for 20 min allowed for the system to equilibrate. An electrochemical workstation measured the open circuit potential of the SE vs. an Ag/AgCl RE with a sampling rate of 10 Hz. The characterization of electrochemical impedance spectroscopy exploited a three-electrode set up with a Ag/AgCl as the RE, a Pt wire as the counter electrode, and K₄Fe(CN)₆/K₃Fe(CN)₆ (1:1) (1 mM for both) as the redox couples. All measurements in this study took place at room temperature.

Deposition of Pd nanoparticles for pH regulation. Treating an Au electrode using cyclic voltammetry (from −0.7 to 0.5 V vs. Ag/AgCl) in 1 wt. % Pd(NO₃)₂ for 10 cycles deposited a layer of Pd nanoparticles (PdNps) on the surface. Loading PdNps in an acidic solution (pH=4.0) with an applied voltage of −1.0 V for 200 s converted the surface to PdHₓ. Placing the Pd/PdHₓ electrode into a pH-neutral solution and applying a positive voltage of 0.8 V enabled the release of protons. A pH indicator (50 μL per 1 mL solution) visualized the pH gradient at the solution-electrode interface and a camera recorded the dynamic change of the gradient as a function of time.

Characterization of deposited PdNps. A Thermo Scientific Apreo FEG SEM characterized the surface morphology of bare gold film and gold film deposited with PdNps before and after the load of hydrogen (acceleration voltage: 5 kV). EDS analyzed the elemental composition of the surface before and after deposition of PdNps.

Fabrication of stretchable “flower-shaped” sensing/actuation system. The patterning of metal traces of the electronic device followed a cut-and-paste method. The process began with laminating a conductive copper tape (Amazon, for the inductive coupling unit) or a polyimide (PI) (HD MicroSystems) film (thickness: (˜13 μm) with 300 nm Au onto an ultraviolet (UV) light dicing tape (Shenzhen You-San Technology Co.). Cutting through the conductive film with a vinyl cutter (Silhouette Cameo 4), exposing the system to UV light, and peeling off unneeded parts created patterned metal traces. Binding the top side of the patterns to a Dragon Skin (Smooth-On) substrate and peeling off the UV releasable tape completed the transfer of the conductive traces. Laminating another layer of Dragon Skin encapsulated the whole system after the soldering of all the electronic components.

Silver epoxy paste electrically connected the copper tape and the stretchable Au serpentine wires. Manually wrapping coils and soldering electronic components to the patterned copper traces according to the circuit diagram completed the fabrication of this stretchable electronic system. This study used the following electronic components assembled as shown in FIG. 21: a pair of varactor diodes (SMV1249) and a damping resistor (10 Kohm) for the sensor (coil diameter: 3 mm, 5 turns); two capacitors (15 pF connected to coil and 1 μF connected to bridge rectifier), a bridge rectifier (BAS3007A-RPP), a Zener diode voltage regulator (CMOZIL8), and a resistor (490 ohm) for the electrochemical actuator (coil diameter: 5 mm, 20 turns).

Preparation of thin-film Ag/AgCl RE. Drop-casting a mixture of silver epoxy and hardener (Chemtronic CW2400), curing at room temperature for 12 hours and transforming the surface into AgCl by treating it with sodium hypochlorite solution (5 wt %) for 30 min formed the thin-film Ag/AgCl RE. Meanwhile, preparation of recrystallized KCl (aq) in cold isopropyl alcohol (IPA) yielded ultrafine micro-size powders. Dissolving 438 mg polyvinyl butyral (PVB, 10 wt %) in 5 mL anhydrous ethanol, mixing the solution with 250 mg KCl powder, and homogenizing the system in an ultrasonic bath for 10 min yielded an electrolyte cocktail (stored at 7° C.). Drop-casting the cocktail on the Ag/AgCl electrode followed by drying overnight completed the fabrication of the RE.

Characterization of wireless electronic system. The reader electronics used for the characterization of resonance frequency consisted of a NanoVNA with a single turn primary coil (diameter: 3 mm) connected through a Sub-Miniature Version A (SMA) connector. Vertically aligning the primary coil with the electromagnetic coupling unit and sweeping the frequency range obtained the real and imaginary parts of the reflection coefficient (S11) with a dip in the resonance curve around fₛ. Fitting the curve determined the value fₛ for quantitative analysis.

To characterize the actuation system, an incident RF power was provided by a function generator (Agilent) (V_{AC, sin}=˜10 V/pp, transmitting frequency=13.3 MHz) and enlarged by an amplifier (V_{bias}=0 V, V_{d}=30 V). The RF power is transmitted wirelessly through a transmitter (10 turns, diameter=3 cm) to the receiver with a vertical distance of 5 mm. According to the EIS characterization of the Pd electrode (FIG. 18), a load resistor (10 Kohm) was connected at the output port of the actuator, and the voltage between the load resistor was recorded with an electrochemical workstation.

Mechanical test. A tensile test system (Instron) was utilized to evaluate the mechanical properties of the wireless system. Shorting the cathode and anode stabilized the varactors in the magnetic coupling unit and minimized environmental noises. fₛ of the sensing system was monitored with an applied tensile strain ranging from 0 to 20%. For the cyclic stretching test, the fₛ was monitored before and after 0-1000 stretching cycles with an applied tensile strain of 20%.

Statistical Analysis. For measurement of the OCP response of the potentiometric sensors, data were calibrated by using the reading of OCP vs. RE in solutions with the lowest concentration of cocaine (10⁻¹¹ M) as the baseline (i.e., calibrated response=0 mV) to mitigate device-to-device variation. Data were expressed as mean±standard deviation (SD). The sample size (n) for each statistical analysis was 3 except for those in FIGS. 3(E), 3(F), 3(G) and 20.

The sensors, devices, and methods of the appended claims are not limited in scope by the specific sensors, devices, and methods described herein, which are intended as illustrations of a few aspects of the claims. Any sensors, devices, and methods that are functionally equivalent are intended to fall within the scope of the claims. Various modifications of the sensors, devices, and methods in addition to those shown and described herein are intended to fall within the scope of the appended claims. Further, while only certain representative sensors, devices, and methods steps disclosed herein are specifically described, other combinations of the sensors, devices, and methods also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

1. A sensor for detecting an analyte of interest, the sensor comprising
a potentiometric sensor comprising a surface functionalized with a pH-sensitive aptamer switch that specifically binds the analyte of interest, wherein the pH-sensitive aptamer switch is operatively coupled to the potentiometric sensor such that binding of the analyte of interest by the pH-sensitive aptamer switch induces a measurable change in the potentiometric sensor; and
an auxiliary electrode in proximity to the surface, wherein the electrode is configured to alter a pH of a microenvironment in contact with the surface, thereby reversibly shuttling the pH-sensitive aptamer switch between a first state wherein it specifically binds the analyte of interest and a second state wherein it does not specifically bind the analyte of interest.

2. The sensor of claim 1, wherein the pH-sensitive aptamer switch is covalently conjugated to the surface.

3. The sensor of claim 1, wherein the surface comprises an electrode surface or a surface of a channel in a field-effect transistor.

4. The sensor of claim 1, wherein the auxiliary electrode comprises a Pd electrode.

5. The sensor of claim 1, wherein the analyte of interest comprises a ligand, small molecule, ion, salt, metal, enzyme, drug, nanoparticle, environmental contaminant, toxin, fatty acid, steroid, hormone, carbohydrate, amino acid, peptide, microbe, virus, nucleic acid, or biomolecule.

6. The sensor of claim 1, wherein the analyte of interest comprises a biomolecule.

7. The sensor of claim 1, wherein the analyte of interest comprises a small molecule (e.g., an organic molecule having a molecular weight of less than 1,000 Da, less than 800 Da, or less than 500 Da).

8. The sensor of claim 1, wherein the analyte of interest comprises a drug, such as cocaine.

9. The sensor of claim 1, wherein the analyte of interest comprises a neurotransmitter, such as serotonin or dopamine.

10. The sensor of claim 1, wherein the pH-sensitive aptamer exhibits a binding affinity for the analyte of interest in the first state that is at least 10, 50, 100, 250, 500, or 1000 times greater than a binding affinity for the analyte of interest in the second state.

11. The sensor of claim 1, wherein the potentiometric sensor is inductively coupled to a signal transducer, a transmitter, or a combination thereof.

12. The sensor of claim 1, wherein the sensor is biocompatible.

13. The sensor of claim 1, wherein the sensor is implantable.

14. The sensor of claim 1, wherein the sensor is flexible.

15. The sensor of claim 1, wherein the sensor is integrated into a regeneratable wearable electronic device, a bioimplant, a point of care diagnostic, or any combination thereof.

16. A method of detecting an analyte of interest in a medium comprising contacting the medium with the sensor of claim 1.

17. The method of claim 16, wherein the medium comprises a biological sample, such as bodily fluid or tissue.
